(19) United States
(12) Patent Application Publication
(10) Pub. No.: US 2024/0207836 A1
(43) Pub. Date: Jun. 27, 2024

(54) SYSTEM INCLUDING HAND-HELD PIPETTING DEVICE

(71) Applicant: EPPENDORF SE, Hamburg (DE)

(72) Inventors: Lisa Marie Timmermann, Handeloh (DE); Benjamin Förthmann, Hamburg (DE)

(21) Appl. No.: 18/557,017

(22) PCT Filed: Apr. 29, 2022
(86) PCT No.: PCT/EP2022/061474

§ 371 Date: Oct. 24, 2023

(30) Foreign Application Priority Data
Apr. 30, 2021 (EP) 21171551.1

(51) Int. Cl. B01L 3/02

(52) U.S. Cl. CPC B01L 3/0237; B01L 2300/023; B01L 2300/027

(57) ABSTRACT

The invention relates to a system for logging a sequence of pipetting operations that are components of a pipetting method used in a laboratory experiment, the system implementing an interruption of a method recording and the possibility of its continuation. The invention also relates to a method of using the system.

The invention relates to a system comprising at least one hand-held pipetting device, and a method of using the same.

Such hand-held pipetting devices are commonly used in medical, biological, biochemical, chemical and other laboratories. They are used in the laboratory for transporting and transferring fluid samples with small volumes, especially for precise dispensing of samples. Two known classes of such hand-held pipetting devices differ in the respective physical principle of fluid aspiration or dispensing. Either the liquid is dispensed by the air cushion principle or the direct displacement principle is used. The first class of devices is called air cushion pipettes, the second class is called direct displacement pipettes.

Air cushion pipettes use a piston-cylinder system with which the actual measurement takes place. An air cushion separates the sample aspirated into a plastic tip from the piston inside the pipette. As the piston slides up, a vacuum is created in the tip, causing the liquid to rise into the tip. The air cushion moved by the piston acts like an elastic spring from which the volume of liquid in the tip hangs. In hand-held pipetting devices that operate according to the direct displacement principle, tips with an integrated piston are used as transfer vessels. This piston is coupled to the piston rod of the dispensing device during the pipetting operation and takes over the actual dispensing operation. Since the piston in this case can displace the entire internal volume of the tip, essentially no air cushion is formed between the aspirated sample and the end of the piston. Both classes of handheld pipetting devices, the air cushion pipettes and the direct displacement pipettes, are also referred to as piston-stroke pipettes.

There are hand-held pipetting devices whose piston system is driven manually and those with electric drive. An electrically driven, hand-held piston-stroke pipette is often controllable by at least one pipetting program to perform at least one type of pipetting operation in an automated or semi-automated manner.

Hand-held pipettes are designed for one-handed use by human users. However, there are also laboratory automats with robotic gripper arms whose gripper tools simulate the activities of a human hand for operating a hand-held pipette and which are set up for handling and operating a hand-held pipette.

In a pipetting device, the sample quantity dispensed by a single actuation can correspond to the sample quantity aspirated into the device. However, it can also be provided that a sample quantity taken up corresponding to several dispensing quantities is dispensed again step by step. In addition, a distinction is made between single-channel pipetting devices and multi-channel pipetting devices, wherein single-channel pipetting devices contain only a single dispensing/receiving channel and multi-channel pipetting devices contain several dispensing/receiving channels, which in particular permit the parallel dispensing or receiving of several samples.

The hand-held pipetting devices described in the context of the present invention are preferably, but not exclusively, hand-held, computer-controlled piston stroke pipettes with electric piston drive, also referred to as hand-held, electric pipetting devices or hand-held, electric piston stroke pipettes.

An example of a hand-held, electronic, prior art air cushion pipette is the Eppendorf Xplorer® and Xplorer® plus from Eppendorf AG, Germany, Hamburg; examples of handheld, electronic dispensers are the Multipette® E3 and Multipette® E3x from Eppendorf AG, Germany, Hamburg.

Electric pipetting devices offer numerous advantages over non-electric pipetting devices, as a wide range of functions can be implemented in a simple manner. In particular, electric pipetting devices can simplify the execution of certain program-controlled pipetting operations by automating or partially automating them. Typical pipetting parameters for controlling such pipetting operations by means of corresponding pipetting programs relate to the volume when aspirating or dispensing liquid, their sequence and repetitions, and, where appropriate, their temporal parameters when distributing these operations over time. An electric pipetting device may be adapted to be operated in one or more operating modes.

An operating mode may provide that a set of one or more pipetting parameters of the pipetting device that affect or control a pipetting operation of the pipetting device is automatically interrogated, set, and/or applied.

Hand-held pipetting devices are known in which a touch-sensitive screen is used to allow the user to enter pipetting parameters, based on which a pipetting operation can be performed by the hand-held pipetting device in an at least partially automated manner.

A convenient pipetting system is marketed under the name “VisioNize® pipette manager” or “Connected Pipettes” by Eppendorf AG, Hamburg, Germany. A productive and efficient working environment is created by combining hand-held pipetting devices with a portable PC that communicates wirelessly with them. The portable PC can be used to configure the pipetting devices, in particular to define pipetting parameters for one or more pipetting operations. The portable PC is an operating module with a touch screen. At the heart of the functionality is a software program, referred to as “VisioNize® pipette manager”, which allows the configuration of one or more hand-held pipetting devices via wireless data link. With this system, several pipetting devices of the same type or different types can be controlled.

An important aspect in the efficient performance of laboratory work is the reproducibility of pipetting methods performed in experiments involving a sequence of pipetting operations performed on laboratory samples. In said pipetting system, a documentation function has been introduced, which is the basis of the present invention.

The present invention is directed to providing a system for recording pipetting operations in user-defined experiments.

The invention solves this problem in particular by means of the objects of the independent claims. Preferred embodiments are the subject matter of the subclaims and can also be found in the description.

The invention relates to a system for recording pipetting operations in user-defined experiments in which at least one hand-held pipetting device is used to pipette at least one sample. An experiment includes a method defined by a sequence of pipetting operations. A set of pipetting parameters defines a pipetting operation. A pipetting operation is a sequence of steps performed automatically or semi-automatically—often requiring one or more trigger inputs from the user—by a pipetting device, and in particular involving the electronic/software-controlled or manually controlled movement of a moving member, in particular a piston, through which one or more liquid laboratory samples are pipetted. “Pipetting” in this context includes: aspirating at least one liquid sample into at least one pipetting container, in particular a pipette tip or dispensing syringe; subsequently holding the sample in the at least one pipetting container; and dispensing the sample from the at least one pipetting container into one or more target containers. The pipetting operation defined by the pipetting parameters may be associated with an operating mode of the pipetting device. A pipetting device may provide a variety of operating modes to assist the user in a variety of application scenarios. These will be explained further below.

Activities carried out in laboratories in which pipetting operations are used follow a sequence that can ultimately be individually specified or at least influenced by each user. The sum of the activities performed by a user to process one or more liquid samples starting from an initial state to a final state is called an experiment. Typically, not only a number of samples are used, but also a variety of different pipetting devices and pipetting containers compatible therewith, which in particular may have different nominal volumes. In an experiment, efficiency is considerably improved by a logging function if the individual pipetting operations do not have to be recorded manually by the user in a lab book but are recorded electronically/software-controlled.

The method is preferably not defined (fixed) in advance. The special feature is that individually arising methods can be recorded. This makes the use of the system or method according to the invention functional and flexible. This is especially true when compared to prior art systems where an experiment must always follow a predetermined protocol. In the present invention, the method record itself can be used to define the method, which can then be made available for subsequent re-use or other users. This goes beyond the mere suitability of the method record as a documentation tool.

The method is preferably defined only by its execution and recording by the executing user. In particular, it is not defined beforehand and, in particular, is not executed according to a predefined plan. The user defines the method by his actions, i.e. by selecting and executing pipetting operations. In this respect, the method is not predetermined. In particular, it is not predefined by a pre-programmed definition and/or sequence of pipetting operations.

An experiment contains a method which is defined by a sequence of pipetting operations. As long as the method is still being defined, it is preferably considered as not yet defined. The method is defined when the parameters required to reproduce the method are stored in a data storage device of the system, in particular the pipetting parameters required to define the pipetting operations of the method and the sequence of the pipetting operations, in particular the order in which they are to be performed and/or a timing of the sequence. The method is finally defined when the user executing the method terminates the method by input to the user interface device, thereby terminating the method recording, and the method is finally known and defined. In particular, an experiment involves a method that is not known in advance and is defined only by executing a sequence of pipetting operations.

The method is preferably user defined and in particular not predefined.

In a more general embodiment of the system according to the invention and the method according to the invention, it does not matter whether a specific user or changing users or users with the same or different user roles, i.e., with respect to their user rights optionally assigned differently or identically by the data processing device, define and/or execute the method and/or its method recording. In this context, it is possible that no user authentication is implemented, or alternatively that user authentication is implemented. Preferably, the system and/or the data processing device is programmed to define and/or execute a method, in particular the first and/or second method, independently of an identity of the user. Preferably, the system and/or the data processing device is programmed such that the execution of the method in the form of the method record can be documented independently of the identity of the user. By these measures, an appropriately designed system can be used in a particularly flexible manner, since in particular any user of the system can design an experiment and define, record, pause and continue or terminate the associated method.

However, it is also possible and preferred that a user authentication is implemented and that the system or the data processing device is programmed such that a method, in particular the first and/or second method, can be defined and executed depending on an identity of the user. Preferably, the system and/or the data processing device is programmed such that the execution of the method can be documented in the form of the method record depending on the identity of the user. Preferably, the data processing device is programmed to distinguish users of the system, in particular by an authentication method. A user may have user group specific rights; in particular, this user may be an administrator user, or may be a user user, with different user rights assigned to these two roles, according to an assignment list stored in a data storage device of the system. The assignment list preferably records, with respect to each user role, the corresponding user rights regarding reading and writing files and/or accessing functions of the system.

The method preferably includes a user-defined sequence of pipetting operations whose sequence and/or pipetting parameters can be defined by a user (in particular, the term “user” can be used synonymously with the term “user”). The user defining the sequence of pipetting operations of a method is in particular that user who also executes the method, for example by initiating or starting the pipetting operations of the method by actuating an actuating element of a pipetting device of the system, who also pauses the execution of the first method recording by means of the input at the user interface device, and/or who also continues the paused, first method recording by means of an input at the user interface device, or terminates the paused, first method recording by means of an input at the user interface device. In this regard, as explained, the system or data processing device may be programmed to implement user authentication, or alternatively, to not implement user authentication.

The user defining the sequence of pipetting operations of a method is in particular a user who also executes the method, for example by initiating or starting the pipetting operations of the method by actuating an actuating element of a pipetting device of the system. The user defining the sequence of pipetting operations of a method is in particular that user who also pauses the execution of the first method recording by means of the input to the user interface device. The user is in particular the user who also pauses the execution of the first method recording by means of the input at the user interface device. The user is in particular that user who also continues the paused, first method recording by an input at the user interface device, or ends the paused, first method recording by an input at the user interface device.

The system according to the invention is a system for recording pipetting operations in a user-defined experiment, which in particular contains a method that is not known in advance and in this respect is only defined by the user performing the experiment.

In principle, it is also possible and preferred that in a system according to the invention a method is defined by a method parameter data record which is stored in a data storage device of the system and which is read by the data storage device so that the user executes the sequence of predetermined pipetting operations defined in the method parameter data record. While this loses the advantage of allowing the executing user to define a method by executing it himself, it retains the advantage of documentation through the method record. In this method recording, a series of data can preferably be stored as well, which in particular are not contained in the method parameter data record, e.g. temporal parameters which have been individually selected by the executing user of the predefined method, and/or preferably sensor data, e.g. an angle of inclination of the pipetting device, and/or acceleration data, and/or environmental parameter data (e.g. temperature, humidity), which can originate from the sensor device of a pipetting device or of the system.

It is typical for electric pipetting devices that the required pipetting parameters are defined by the user before execution. For this purpose, a data processing device is programmed to acquire, by means of a user interface device, e.g. a touch screen, parameter values of user-definable pipetting parameters of at least one parameter set defining the at least one pipetting operation to be performed by means of the at least one hand-held pipetting device.

A pipetting device preferably comprises at least one actuating element with which the user can make inputs to the pipetting device, in particular can start a pipetting operation—and/or its partial processes—which is then carried out automatically. The actuations of the at least one actuating element in the course of a pipetting operation and/or the sum of the events in the course of one or more pipetting operations of a method can be recorded by the data processing device in recording data as well as, preferably optionally in each case, information about at least one of the following: an operating mode used, the pipetting parameters applied during the pipetting operation, start and/or end time of the pipetting operations or their partial steps, identification data—e.g., serial number—of the pipetting parameters used, start and/or end time of the pipetting operations or their partial steps. e.g. serial numbers—of the individual pipetting devices used, identification data of the—optionally previously authenticated or selected-user, who will usually be the executing responsible person of an experiment, as well as other data, which may be provided by other devices and/or sensors of a laboratory involved in the experiment.

The documenting function is implemented in the system by providing a method recording as a system feature, which is implemented by means of a correspondingly programmed data processing device. If this method recording is designated as “first method recording”, this serves only a naming, which permits the reference back to this system characteristic. Internally in the system, the first method record can be referenced by “first method identification data”. The terms “first” and “second”, for example also “third”, etc., which are used in the context of this description to designate items, are typically to be understood as naming, and are generally not used to designate an order or naming of a number, unless this results as applicable in the respective context. In particular, the naming of “first method record” and “second method record” does not imply that a third and further method record is not possible; the designation “first” and “second” in this context serve primarily to distinguish the method records.

The invention expressly provides that a plurality of method recordings may also be performed in parallel. The data processing device may be programmed to process parameter values from multiple pipetting operations occurring substantially in parallel and, in particular, to store them in the same method record or in different method records. For example, in an exceptional case, a user could operate a particular pipetting device with one hand at a time, and its parameter values could be transmitted substantially in parallel to the first communication device to be stored in the same method record or in different method records. This type of parallel recording is not to be confused with the interleaved recording enabled by the system according to the invention.

The data processing device is preferably programmed to register at least one pipetting device for use with the first method, in particular by storing identification data, in particular a serial number of the pipetting device, in particular together with the first method identification data in the first method data set.

The data processing device is preferably programmed to check, when registering a pipetting device for use with the first method, on the basis of the identification data of the pipetting device, whether the pipetting device is already registered in another running—i.e. started and not yet finished—method record, and in particular, if this is the case, not to register the first pipetting device for use with the first method. In this way, a pipetting device will only be associated with exactly one running method recording, which prevents confusion on the part of the user and improves the safety of working with the system.

On the one hand, pausing a method recording allows undocumented work, i.e. performing pipetting operations with at least one pipetting device that is or was registered for use with the first method—at least before the recording pause. On the other hand, this at least one pipetting device can be assigned to another method recording started or running during the recording pause by registration, which allows a more efficient use of existing pipetting devices under substantially continuous use of a documentation function. This type of interleaving of method recordings is therefore a preferred further development of the invention.

During the first method recording, in particular, a first sequence of parameter sets corresponding to a first sequence of pipetting operations performed by means of the at least one hand-held pipetting device used in a first experiment is recorded in the data storage device. In a first recording portion of the first method recording, in particular, a first subset of the first sequence of pipetting operations is stored in the first method data set. In a second recording portion of the first method recording, in particular, a second subset of the first sequence of pipetting operations is stored in the first method data set. In a third (i-th, i a natural number) recording portion of the first method recording, in particular a third (i-th) subset of the first sequence of pipetting operations is stored in the first method data set. In particular, there is a recording pause between each of the recording portions. The pipetting operations of a method recording are thus interrupted, in particular by one or more pauses, successively collected and stored in the first method data set. The pause duration is not limited in time and depends on the user procedure.

The system according to the invention or the documenting function is preferably characterized in that the method recording can be paused. This does not exclusively mean that a time offset exists between the time intervals of the recording of recording portions of a method recording. Rather, it is preferably provided that a user actively pauses the method recording by manual input at the system, in particular at the user interface device, namely in dependence on a user input at the system, in particular at the user interface device. This allows the user in particular to perform one or more pipetting operations, or other events—e.g., configurations—can be performed on the pipetting devices of the system, or on the pipetting devices registered for use with a method, which are then not recorded in the method recording. After the pause, the method recording may optionally be continued or terminated. The data processing device is preferably programmed so that in the status of the pause—for the user optionally—a continuation of the first method recording or its termination is possible.

The phrase “in response to a user input” means that a user input made via the user interface device is detected, and optionally that its content is evaluated, and means that when that user input is detected, method recording is started, paused, or continued, depending on the context of the phrase “in response to a user input.”

Pausing (also referred to as: stopping) is not the same as stopping method recording. The pausing option gives the system a previously unknown flexibility in performing experiments and logging them in laboratories. Preferably, the data processing device is programmed so that no further recording data, in particular parameter values, can be added to a method recording that has been given the status “terminated” by termination by storing it in the first method data set. For example, however, a verification value can be assigned to the first method data set after termination of the first method recording in order to exclude undetected manipulation of the first method data set after termination of the first method recording.

Preferably, the data processing device is programmed to:
a) start, in response to a user input, the recording of a first recording portion of the first method recording, wherein a first subset of the first sequence of parameter sets of the first experiment section is recorded and stored as a first method data set in the data storage device,
b) pause, in response to a user input, the first method recording.
Preferably, it is then provided: c) in response to a user input and in response to the first method identification data, to select the stored first method data set and to terminate or continue the first method recording the first experiment, in that in the case of continuation, the recording of a second recording portion of the first method recording is started, in which the second subset of the first sequence of parameter sets of the second experiment section is recorded. In the case of termination of the first method recording, the data processing device is preferably programmed to prevent further continuation of the first method recording and/or, in particular, subsequent modification thereof.

The continuability of the first method recording results in numerous advantageous application scenarios of the logging: A number N>1 of methods can be nested, executed in parallel and also recorded. For this purpose, the first method recording can be paused, a second method recording can be continued or started, the second method recording can be paused, a third method recording can be continued or started, the third method recording can be paused, and so on.

A method for using the system may in particular provide for the following successive mode of operation, including in particular the following steps to be performed successively, which may be components of a recording portion:
a1) Configuring at least one pipetting device by at least a first set of parameters specified by the user at the user interface device;
a2) Performing at least one pipetting operation according to the configuration of the at least one pipetting device by the at least one first parameter set;
a3) Record the parameter values of the first parameter set in the first method data set;
a4) Configuring at least one pipetting device by at least a second set of parameters specified by the user at the user interface device;
a5) Performing at least one pipetting operation according to the configuration of the at least one pipetting device by the at least one second parameter set;
a6) Record the parameter values of the first parameter set in the first method data set;
in particular, the following sequence can be repeated several times or as often as desired—and for any method recording—(j=1 . . . n; n a natural number):
a_i) Configuring at least one pipetting device by at least a j-th set of parameters specified by the user at the user interface device;
a_ii) performing at least one pipetting operation according to the configuration of the at least one pipetting device by the at least one j-th parameter set
a_iii) Record the parameter values of the jth parameter set in the first method data set.

Alternatively to a successive configuration of the pipetting operations, the data processing device could be programmed to determine a sequence of parameter sets defined by the user at the user interface device, by means of which at least one pipetting device is configured for a plurality of successive pipetting operations, wherein the corresponding recording data can be transmitted successively—e.g. after completion of a respective pipetting operation defined by a parameter set—to the first communication device for the purpose of recording, or wherein the recording data can be transmitted not successively but continuously, for example at the end of a method or a recording portion, to the first communication device for the purpose of recording.

The method record(s) can be assigned to one and the same user or to different users. Since the method records are interleaved in time, this enables the use of one and the same pipetting device—e.g. identified by its identification data—in several of the interleaved method records.

In a typical application scenario, a user performs an initial method on initial samples in a first experiment—e.g. dividing a stock solution—e.g. a cell suspension—into individual containers, distributing the parts into individual tubes, adding liquid to be mixed into each individual tube. Then centrifugation of the samples in the individual tubes may be necessary. This creates a waiting period within the first experiment. By pausing the first method recording, the user can start a second experiment, also recorded, in particular using the same pipetting devices already used in the first method and assigned to it, in particular by registration, without having to take an unproductive break from work. In principle, the number N of nested recordable methods is not limited, in particular N>5, N>10 and especially N between 1 and 100.

The data processing device is preferably programmed to perform an authentication method in which a user is logged into a session and user identification data is determined as a function of this user, in particular by selection from a collection of existing user identification data or by assignment of newly determined, individual user identification data. In particular, method recording is performed in dependence on user identification data, and in particular, user identification data is stored with the method recording in the data storage device.

The data processing device is preferably programmed to distinguish users of the system, in particular by means of an authentication procedure. This procedure is considered to be part of an optional user management, in particular one that can be activated and/or deactivated by a user, whereby this user may have user group-specific rights; in particular, this user may be an administrator user, who may have the right to activate and/or deactivate the user management with respect to a standard user. When user management is enabled, only the respective user can access and continue a method assigned to him. If user management is deactivated, different users can access (and thus continue) a method. Ex: Lab employee 1 starts method in the morning, lab employee 2 continues the method in the afternoon.

A hand-held pipetting device for pipetting at least one liquid sample can be a piston-stroke pipette operating according to the air cushion principle or a repetitive pipette operating according to the direct displacement principle, also referred to as a dispenser. The handheld pipetting device has in particular:
a connecting portion for connecting at least one pipetting container;
an electrically controlled moving member for aspirating at least one sample into with the at least one pipetting container, holding the sample in the at least one pipetting container, and dispensing sample from the at least one pipetting container when performing a pipetting operation; and
an electrical control device arranged to control the moving member in dependence on the at least one pipetting parameter;

Preferably: * a communication device for receiving the parameter values of the user-definable pipetting parameters from a computing device, in particular a portable PC, in particular a tablet PC, located remotely from the pipetting device.

The, in particular portable, computer device preferably comprises, in each case:
this user interface device with the display,
this data processing device,
optionally: the first communication device for sending the parameter values of the user-definable pipetting parameters to the at least one second communication device of the at least one hand-held pipetting device, and
optional: this data storage device,
a housing in and/or on which said components are arranged, and which can be held in particular by a user with one hand and the computer device can be operated in particular with one hand.

The portability of the components makes the system particularly flexible and easy to use in the laboratory.

Instead of a portable computer device, a non-portable computer device, e.g. a stationary PC, can also be used with otherwise the same functionality. The user interface device with the display can in principle also be a component of a pipetting device.

Preferably, the data processing device is programmed to detect, after pausing the first method recording in step b), by means of the user interface means at least one further parameter set of at least one further pipetting operation which does not form part of the first sequence of pipetting operations, and wherein in particular the control means is arranged to control, after pausing the first method recording in step b), the moving member in dependence on the at least one further parameter set. By this step the advantage is realized that pausing the method recording also allows pausing a logged experiment, whereby during the pause productive work can be continued in the form of the further pipetting operation, which can at least be configured by the user or also actually executed by the user.

Preferably, the data processing device is programmed to perform a second method recording after pausing the first method recording in step b) and before starting the recording of the second recording portion of the first method recording in step c), to which second method identification data (different from the first method identification data) are assigned and which records in the data storage device a second sequence of parameter sets corresponding to a second sequence of pipetting operations performed by means of the at least one hand-held pipetting device and used in a second experiment, in particular also records time data of these pipetting operations. By this step, the advantage is implemented that during a pause of the first experiment, a second experiment can be started and also recorded without disturbing the recording of the first experiment, which can be continued or stopped later. Similarly to the case where a second experiment (a second method) is started after step b), a second experiment can also be continued provided that it has already been started, its recording already started and paused in the meantime. The number of nested experiments or method recordings is not limited to a “first” and a “second” method recording, as already emphasized; any number of methods/experiments can be recorded in a nested manner.

Preferably, the data processing device is programmed to display on the display of the user interface device
display a screen page with a button, the pressing or touching of which starts a method recording.

Preferably, the data processing device is programmed to display on the display of the user interface device
display at least one screen page in which the title of at least one running method recording is displayed, in particular in which the titles of several running method recordings are listed,
display at least one information field indicating the status of the corresponding method recording, which may include: Recording is paused or Recording is running or Recording is finished.

Preferably, the data processing device is programmed to display on the display of the user interface device
display a screen page in which the title of at least one running method recording is displayed, in particular in which the titles of several running method recordings are listed,
display at least one button whose press or touch selects the at least one previously paused method record associated with the button;
and to display a button whose actuation or touch resumes the selected paused method recording, in particular while at least one other method recording is paused.

Preferably, the data processing device is programmed to display on the display of the user interface device
display a screen page in which the title of at least one running method recording is displayed, in particular in which the titles of several running method recordings are listed,
display at least one button whose press or touch selects the at least one previously paused method record associated with the button;
and display a button whose pressing or touching resumes the selected paused method recording.

Preferably, the data processing device is programmed to provide at least one method data set of at least one terminated method record as a file transferable to an external data processing device, or the contents of which are outputtable by printing, displaying on screen, or transferring to an external data processing device of the system or a server that is not part of the system.

The data processing device is preferably programmed to capture one or more note input(s) performed by the user via the user interface device during the performance of a method recording and to store them as part(s) of the corresponding method data set with this method data set. The note input preferably includes a text individually entered by the user, in particular by a virtual keyboard—or alternatively by a (hardware) keyboard actually associated with the, in particular portable, computing device. However, the note input may also—alternatively or additionally—include that a predefined selection list of possible note texts is provided to the user on the screen, from which the user can then select the appropriate note text. The content of such note texts can relate to frequently occurring typical situations, e.g. a technical error observed by the user during the pipetting operation or a user-caused error or other influencing factor, e.g. environmental factor: temperature, humidity, or sample condition, which as an undesired or desired factor could in particular affect the results of the experiment.

The one or more note inputs can each preferably be stored with a time stamp, which in particular enables a subsequent assignment of the one or more note inputs to other partial steps of the pipetting operations of the method or generally, to another recorded event (event) of the method recording and/or enables the chronological classification of the note. The one or more note inputs can each preferably also be stored without their own time stamp; preferably, a note input is stored in such a way that an association of the note input with another event contained in the method data set or the association with a group of events of the method data set is also stored. In this case, this other event or group of events preferably has at least one time stamp. This also makes it possible to classify the note input in terms of time.

The data processing device is preferably programmed to output the event history, also referred to as “event log”, of a method data set, i.e. the sequence of events forming the method recording, on a screen—in particular of the user interface device—, in particular as a list or sequence of these events, with each event preferably being displayed with a corresponding descriptive text and/or a graphic symbol. The event log can be used as a protocol of the performed experiment or method recording.

When automated pipetting operations are mentioned, semi-automated pipetting operations are always also meant, unless the respective context contradicts this. Performing an automated pipetting operation always includes at least one user activity that triggers at least the automated pipetting operation, usually by actuating an operating element of the pipetting device. Partially automated pipetting operations of certain application scenarios may, for example, require that a user, in addition to starting a pipetting operation divided into partial operations, at least also starts the triggering of a partial operation by actuating an operating element. The automated pipetting operations of a particular application scenario may, in certain embodiments of the pipetting device, be defined by a set of pipetting parameters that the user sets before starting an automated pipetting operation by means of the operating device of a user interface device of the pipetting device, and/or adopts from default settings and/or loads from a memory. Typically, the user interface of such embodiments of the pipetting device has a selection option, for example a control wheel or a list of selectable fields on a touchscreen, by means of which a so-called “operating mode” is selectable, representing the specific application scenario, or in which the whole set of pipetting parameters is selected, belonging to this specific application scenario, and which the user can set before starting the associated automated pipetting operation. The automated pipetting operation then runs according to a pipetting program of this operating mode specified as an operating program.

In a preferred embodiment, the system, in particular the pipetting device or data processing device, is programmed to allow the user to manually define an automated pipetting operation by selecting pipetting parameters, and thus in particular to define a set of pipetting parameters (pipetting parameter set) of a user-defined pipetting operation. This pipetting parameter set can correspond partially or completely to a predefined pipetting parameter set of an operating mode. Pipetting parameters of a pipetting operation preferably concern or quantify the volume to be pipetted at the step of aspirating the sample into a pipetting container connected to the piston-stroke pipette or at the step of dispensing the sample from this pipetting container, optionally the sequence and repetitions of these steps, and optionally temporal parameters in the temporal distribution of these operations, in particular also the temporal variation of such operations, in particular the speed and/or acceleration of the aspiration or dispensing of the sample. In this embodiment, the automated pipetting operation runs according to a pipetting program that is fully defined according to these user-defined pipetting parameters.

The hand-held pipetting device according to the invention is preferably designed to be used to perform at least one pipetting operation according to at least one parameter set of at least one, or at least two, or at least three pipetting parameters, in particular to be used in at least one predetermined operating mode according to a predetermined parameter set of the pipetting device or in a user-defined parameter set. In an operating mode, one parameter set of pipetting parameters (pipetting parameter set) is preferably provided in each case, the pipetting parameters of which are optionally not selectable by the user or are optionally at least partially deselectable by the user and can be supplemented by further pipetting parameters.

A pipetting operation typically provides that, in accordance with a pipetting program, a specific sample quantity is taken up from a start container into a pipetting container, in particular a pipette tip, connected to the piston-stroke pipette, and/or is dispensed into a target container, in particular is dispensed in a metered manner. A pipetting operation can preferably be controlled by at least one, two, three or preferably several pipetting parameters, with which the said pipetting operation, or a function or component thereof, can be influenced or defined in the desired manner.

Pipetting parameters for controlling a pipetting operation relate to or preferably quantify the volume to be pipetted, in the step of aspirating the sample into a pipetting container connected to the piston-stroke pipette or in the step of dispensing the sample from this pipetting container, optionally the sequence and repetitions of these steps, and optionally temporal parameters in the temporal distribution of these processes, in particular also the temporal variation of such processes, in particular the speed and/or acceleration of the aspiration or dispensing of the sample, or the number of pre-wetting steps and optionally the volume aspirated and dispensed in each case.

These pipetting parameters are preferably at least partially and preferably completely selected and/or entered by the user, in particular via the at least one operating element of the user interface device of a piston-operated pipette or an external data processing device.

The pipetting operation is preferably uniquely definable or defined by the pipetting parameter set. This pipetting parameter set is preferably at least partially and preferably completely selected and/or entered by the user, in particular via the operating device of the pipetting device or the external data processing device.

However, it is possible that a pipetting operation is not unambiguously defined by the pipetting parameter set; in this case, the pipetting device is in particular set up or the data processing device is programmed to automatically supplement the pipetting parameter set with at least one further pipetting parameter, in particular as a function of the (selected) pipetting parameters. It is possible and preferred that at least one pipetting parameter is not defined by the user, but is predefined by the pipetting device, for example, by being stored there in a data memory in a previously known manner or by being determined automatically.

Preferably, at least one pipetting parameter is provided with which the number of directly successive or indirectly successive pipetting volumes is determined, preferably at least one pipetting parameter with which the number of aspiration steps and/or dispensing steps and in each case preferably also the respectively associated pipetting volumes, the respectively associated pipetting speeds and/or accelerations, and/or the respectively associated time intervals between the steps are determined.

Typical pipetting parameters that can be used for manual definition of user-defined pipetting operations can be taken from the following description of typical application scenarios of automated pipetting operations. Each application scenario can be preset by a correspondingly designated pipetting mode. A pipetting mode can be assigned a pipetting mode ID. The pipetting parameters of a pipetting mode can be set at the user interface device—an appropriately designed GUI, see figures—by the user, the pipetting device can optionally receive the pipetting parameters together with the pipetting mode ID and set the pipetting mode accordingly at the pipetting device.

A typical application scenario envisaged for pipetting devices as a partially automated process is the dispensing (briefly referred to as “DIS”) of a volume received into the pipetting container (pipette tip or dispenser tip). Each partial dispensing, e.g. dispensing a total sample of 1 ml from a pipetting container into a total of ten separate target containers of a microtiter plate, requires the user to position the pipetting device above a reservoir of sample liquid to be dispensed, immerses the tip of the pipetting container into the supply liquid, takes up a starting volume by actuating an operating element, triggers a reverse stroke by actuating an operating element or automatically, which brings the system into a defined starting position, positions it above the first target container, starts the electrically driven delivery of a dosed partial volume of 0.1 ml by actuating an operating element, moves and positions the pipetting device above the next target container of the microtiter plate, starts again the electrically driven delivery of a dosed partial volume of 0.1 ml by actuating an operating element, and repeats these manually performed partial processes (positioning, triggering), followed by the automated partial process of the electrically driven partial delivery of 0.1 ml sample (of a total of 10 steps of the partial delivery) eight more times. The metered dispensing of the partial volumes is carried out according to the specification of pipetting parameters including one, several or all of the following pipetting parameters: the total volume of the sample to be dispensed, the partial volumes of the sample to be dispensed and/or the number of dispensing steps for dispensing the same partial volumes, the sample uptake speed and/or a possibly deviating sample dispensing speed, the volume of a reverse stroke, the volume of an overstroke, possibly the volume and the repetitions of prewetting steps of the pipetting containers, in particular pipette tips. The dispensing function is particularly suitable for rapid filling of a microtiter plate with a reagent liquid and can be used, for example, to perform an ELISA.

Preferably, an application scenario relates to “Automatic Dispensing” (ADS) of a sample. Associated operating parameters are preferably, in each case: the volume of the individual sample, concerning the pipetting volume during one of several dispensing steps; the number of dispensing steps; the duration of the time interval according to which the dispensing steps are automatically performed one after the other at constant time intervals—the time interval can define these time intervals or, for example, the delay between the end and start of successive dispensing steps; the speed at which the sample(s) is/are taken up; the speed at which the sample(s) is/are dispensed. This dispensing function is even more convenient for filling a microtiter plate, since the user does not have to repeatedly trigger a dispensing step by actuation, e.g. pressing a key, but dispensing is time-controlled after starting automatic dispensing. The execution of the operating program belonging to such a pipetting parameter set, the automatic dispensing can take place under the condition that the corresponding program takes place at least when an actuating element is continuously actuated, e.g. when the key is continuously held down. This is advantageous, for example, in the case of long dispensing series or reactions in which exact observance of a time window is required. The automatic dispensing function is even more convenient for filling a microtiter plate, since the user does not have to initiate a single dispensing step himself here, but this is done automatically, which can be used, for example, to perform an ELISA.

Preferably, an application scenario concerns the “pipetting” (pip) of a sample. Associated pipetting parameters are in particular: the volume of the sample to be pipetted; the speed at which the sample is taken up; the speed at which the sample is dispensed.

Preferably, an application scenario concerns “pipetting with subsequent mixing” (P/Mix) of a sample. Associated pipetting parameters are preferably: the volume of the sample to be aspirated and/or the sample to be dispensed; the mixing volume; the number of mixing cycles; the speed at which the sample is aspirated; the speed at which the sample is dispensed. The “Pipetting followed by mixing” function is recommended, for example, for pipetting very small volumes. If a dispensing volume <10 μL is selected, it is recommended to mix it into the respective reaction liquid. This is possible by automatically starting a mixing movement after dispensing the liquid. The mixing volume as well as the mixing cycles are defined beforehand. One application for this operating mode is, for example, the dispensing of a liquid that is heavier than water due to its physical properties, the residues of which in the pipetting container, in particular the pipette tip, are then rinsed out of the pipetting container, or the pipette tip, with the aid of the liquid that has already been dispensed. Another application would be the immediate mixing of the dispensed liquid with the presented liquid. This operating mode is advantageous, for example, when adding DNA to a PCR mixing solution.

Preferably, an application scenario concerns the “multiple aspiration” of a sample, also referred to as “reverse dispensing” or as “ASP” for aspiration. Associated pipetting parameters are preferably, respectively: the volume of the sample(s) to be aspirated; the number of samples; the speed at aspiration; the speed at dispensing. The function is used for multiple aspiration of a quantity of liquid and dispensing of the total quantity. Multiple filling of the pipetting container in one process is not intended here. The speed is the same for all samples. In the embodiment, the following preferably occurs: Starting from the basic position, the pipetting device takes up one partial volume at a time by actuating the first type of operating device. After the last partial volume has been aspirated, the pipetting device preferably outputs a warning message, which must be confirmed by the user, preferably by actuating the second type of operating device. With the next actuation of the second type of the operating device, the total volume is dispensed again. For actuation of the first or second kind, the operating device preferably has at least two operating elements, one for inputting an operating signal of the “first kind” to the control device, and one for inputting an operating signal of the “second kind” to the control device. In particular, the operating device can have a rocker, which in particular can be pivoted about an axis perpendicular to the longitudinal axis of the pipetting device, between a first signal triggering position (“rocker up”) for actuation of the first type and a second signal triggering position (“rocker down”) for actuation of the second type.

Preferably, an application scenario concerns the “diluting” (Dil) of a sample, also referred to as “dilution”. Associated pipetting parameters are preferably, respectively: the sample volume; the air bubble volume; the diluent volume; the speed of uptake; the speed of delivery. The maximum diluent volume=nominal volume−(sample+air bubble)). This function is used for the uptake of a sample and a diluent with separation by an air bubble and delivery of the total volume. The speed is the same for all partial volumes. Preferably, the following happens in the execution: Starting from the home position, the pipetting device first picks up the diluent volume, then the air bubble and finally the sample. Each pick-up is preferably triggered separately by an actuation of the operating device of the first type. After that, the total volume is dispensed in one.

Preferably, an application scenario relates to “Sequential Dispensing” (SeqD) of samples. Associated pipetting parameters are preferably in each case: Number of samples (preferably up to a fixed maximum number Nmax of preferably 5<=Nmax<=16, preferably Nmax=10); Individual volume of individual samples; Speed of uptake; Speed of dispensing. This function is used for sequential dispensing of Nmax freely selectable volumes, in this case multiple filling of the pipetting container is preferably not provided. The speed is the same for all samples. The number of samples is preferably the leading parameter for the input of the individual volumes. When entering the volumes, the pipetting device must preferably always check that the maximum volume of the pipetting devices is not exceeded; if necessary, a warning message is output. After all parameters have been entered, the pipetting device picks up the total volume after the first type of operating device has been actuated and picks up an individual volume after the second type of operating device has been actuated. All further processes behave preferably like normal dispensing.

Preferably, an application scenario relates to “Sequential Pipetting” (SeqP) of samples. Associated pipetting parameters are preferably in each case: Number of samples (preferably up to a fixed maximum number Nmax of preferably 5<=Nmax<=15, preferably Nmax=10); Individual volume of individual samples; Speed of uptake; Speed of dispensing. This function is used for pipetting maximum Nmax freely selectable volumes, which are programmed before the start and are fixed in their sequence. The speed is preferably the same for all samples. However, the speed can also be set differently. The sequence of the function corresponds to the pipetting sequence. The previously entered volumes are processed in the programmed sequence. After dispensing, an operating element is actuated, e.g. by pressing a key, to decide whether the next sample should be taken or whether a “blowout”, i.e. a complete, safe blowing out of the sample still contained in the pipetting container by means of an overstroke, should be performed before the next sample is taken and/or whether the pipetting container should be changed.

Preferably, an application scenario concerns “reverse pipetting” (rPip) of samples. Associated pipetting parameters are preferably in each case: the volume of the individual sample; the speed of aspiration; the speed of dispensing; activation of the counter. In this “rPip” function, more than the volume to be dispensed is aspirated. This is achieved by moving the piston down before the liquid is aspirated, namely by actuation of the second type, i.e., e.g., by pressing the key or “rocker down”, to the lower position of a blowout, i.e., an overstroke of the piston that exceeds the position of the piston during a pipetting stroke. When the volume pick-up is started, the pipetting device picks up the volume of the blowout and the set volume. To take out the play in the drive in the dispensing direction, the pipetting device completes an additional stroke, which is immediately dispensed again. This is similar to dispensing, but is preferably performed with automatic delivery of the reject stroke at maximum speed.

In the execution of the “rPip” application scenario, the following preferably occurs: First, the piston of the pipetting device (or dispensing tip) automatically moves to the blowout and stops in the down position. Second, an actuation of the first type of the operating device takes place: piston moves up by the blowout distance and by the stroke for the pipetting volume. Thirdly, the second type of operating device is actuated: the piston moves down the stroke for the pipetting volume and stops before the blowout. Fourthly, two actuations of the second type of operating device are performed: the piston performs the blowout and stops in the lower position. As an alternative to “fourth”, the first type of operating device is actuated: the piston moves the pipetting stroke upwards. The “rPip” mode is particularly suitable for pipetting plasma, sera and other liquids with a high protein content. The “Pipetting” mode is particularly suitable for aqueous solutions. The “rPip” mode is particularly suitable for solutions containing wetting agents in order to minimize foam formation during dispensing into the target vessel. The liquid is aspirated in particular with overstroke (blowout volume). In this case, the overstroke is typically not part of the dispensing volume and is preferably not dispensed into the target vessel. In particular, if the same sample is used again, the overstroke may remain in the tip. If another liquid is used, preferably the overstroke and/or preferably the pipetting container is discarded.

An operating program, in particular a control program, for carrying out the desired pipetting operation is preferably controlled by a pipetting parameter set. The control program can in each case be formed in the form of electrical circuits of the control device, and/or be formed by an executable program code which is suitable for controlling a control device which is program code controllable and is preferably programmable.

The system, the pipetting device, a portable computer device or an external data processing device is preferably designed to automatically check the values of the pipetting parameters (pipetting parameter values) entered by the user and to compare them with an allowed range of the respective pipetting parameter. If the pipetting parameter value entered by the user is outside the permissible range, the entry is either not accepted, a warning is issued on the screen and/or acoustically, or set to a default value, which can be, for example, the minimum value or the maximum value or the last permissible value entered, or an automatically corrected value is used.

Preferably, the user interface device comprises at least one touch-sensitive or non-touch-sensitive screen and/or preferably a number of at least partially predefined display pages (also referred to as “screen pages”) stored in the pipetting device in the form of display page data, which can be displayed in one, or distributed over several screens, preferably full-screen. Preferably, the screen area is substantially rectangular, possibly square.

The data processing device is preferably programmed to display a graphical user interface (GUI) in the screen (synonym: display) of the user interface device. In the description of the GUI, the phrase “the data processing device is programmed to that” is partially omitted below and only the GUI and its mode of operation are described, always meaning that the data processing device is programmed to implement the corresponding GUI and its mode of operation. This implementation is a routine activity for the person skilled in the art.

The GUI at least preferably includes the display of a screen page (in particular: “homescreen”) in the screen in which pipetting parameters of a parameter set of pipetting parameters are displayed. Accordingly, the data processing device is programmed to display in the screen a screen page in which pipetting parameters of a parameter set of pipetting parameters are displayed. The expression “a pipetting parameter is displayed in the screen” means that the currently set value of the pipetting parameter is displayed in the screen. In addition, a description of the pipetting parameter can be displayed, in particular a text description, a characteristic pictogram, or a physical unit of the quantity described by the pipetting parameter, if the pipetting parameter concerns such a unit, e.g. “uL” for a volume. However, the skilled person can also know or learn from the context to which pipetting parameter a displayed value is assigned, even if a description is missing from the screen.

In particular, the data processing device is programmed to cause the screen page to display a home screen, synonymously also referred to as a homescreen (analogously: homepage in Internet browsers). It is preferred that a plurality or plurality of user inputs to the GUI lead back to this homescreen. This homescreen can advantageously be used to display the entire parameter set of a user-defined—or taken from a pre-stored application scenario—pipetting operation and to enable settings of the associated pipetting parameters. The home screen is displayed in particular after the user interface device, in particular the portable computing device, has been switched on, in particular immediately after switching on, it being possible that purely informative pages are still displayed temporally before the home screen, e.g. a company logo, device information, etc. The switching on can be effected by a user operation of the display or an operating element of the portable computer device, or can be effected by the measurement of a sensor, e.g. an acceleration sensor, movement sensor, proximity sensor etc. Such a home screen has proven to be an intuitively graspable center of the operating concept of the pipetting device for the users.

The pipetting device or a data processing device external thereto, in particular the computer device, preferably have a data storage device. This preferably has at least one data memory, in particular a hardware data memory, in particular non-volatile data memory, in particular an EPROM or FLASH memory. It may also have a volatile data memory. However, said data storage device may also be contained in another external data processing device, for example a server, which may be part of the system. This server then implements in particular a file hosting, i.e. a cloud service.

The data processing device may be part of an electrical control device of the system, in particular the pipetting device, the portable computer device or an external data processing device. The portable computing device, in particular the electrical control device, may each preferably comprise: a microcontroller, a CPU, data memory for storing control software: the electrical control device, also abbreviated as control device or control device, preferably comprises a data processing device, in particular comprising at least one central processor (CPU). The data processing device may include a microcontroller. The control device preferably comprises a microcontroller. The control device preferably has at least one memory device or data memory for storing data, in particular pipetting parameters and/or one or more computer programs or computer program codes.

The control device preferably includes at least one control software or control program which uses this at least one pipetting parameter to automatically execute at least one function of the pipetting operation or a part of the pipetting operation or the pipetting operation. The control software or the control program is executed in particular by the data processing device of the control device, in particular by a CPU of the data processing device. The control software or the control program is stored in particular in a data storage device of the device. This data storage device is preferably a non-volatile memory.

The system, in particular the pipetting device, a portable computing device or an external data processing device may include a sensor device, e.g. a sensor for detecting an environmental parameter, in particular the temperature, humidity or pressure, of the motor current used to drive the piston of the pipetting device. In particular, the motor current can be used to determine the viscosity of the pipetted liquid, and thus to identify the liquid. The sensor device can also be designed to perform a measurement that can be used to determine a parameter, in particular pipetting parameters, in particular the type of pipetting container connected to the pipetting device, in particular the maximum filling volume of the pipetting container, in particular of a pipette tip. The pipetting device or an external data processing device can be designed to automatically determine at least one pipetting parameter as a function of the measured value of the sensor device. This makes it possible to improve the optimization of the pipetting parameters required for precise pipetting.

The pipetting device and/or a portable computer device and/or an external data processing device is preferably operated independently of the mains. In particular, the respective device may be provided with a rechargeable voltage source, for example one or more rechargeable batteries. For this case, the device may have a charging interface connected to the rechargeable voltage source.

A communication device, in particular the first or second communication device, is preferably set up for wireless data exchange via a radio network. A communication device, in particular the first or second communication device, is preferably designed for data exchange via WLAN, Bluetooth and/or LoRa-WAN. The system components exchange data in particular by means of such radio technologies. The system components—without the at least one pipetting device—can be arranged in different devices, i.e. in particular housings, or can be arranged in the same housing, in particular the housing of a computer device. In particular, the data storage device may be included in the system as a component of a server for implementing a file hosting service.

Pipetting containers are containers adapted to be connected to a connecting portion of a hand-held pipetting device so as to allow aspiration of liquid into the pipetting container, retention of the liquid in the pipetting container, and dispensing therefrom. Examples of pipetting containers include pipette tips and dispensing tips.

Pipette tips are in particular disposable products and are preferably made of plastic. Depending on the required maximum liquid volume, different pipette tips are used with the piston-stroke pipette. Typical nominal volumes of commercially available pipette tips are, for example, 10 μL, 20 μL, 100 μL, 200 μL, 300 μL, 1000 μL, 1250 μL, 2500 μL, 5 mL, 10 mL (μL: microliter; mL: milliliter). A pipette tip generally has a cone-shaped container elongated along a longitudinal axis, which has a liquid exchange opening at the lower end, and which has a cone- and tube-shaped, upwardly open end section at the upper end, which can be plugged onto a connecting portion designed as a working cone of the pipetting device designed as an air cushion pipette, in particular. Suction of the liquid into the pipette tip is effected via a negative pressure in the interior of the pipette tip, which in the case of the pipette device designed as an air cushion pipette is generated by a movement of the moving member of the pipette device designed as a piston, in that a negative pressure is generated in the air space above the sample in the pipette tip. The interior of the pipette tip is fluidically connected to the pipetting channel of the piston-stroke pipette in a pipetting position, also referred to as the push-on position, in which the pipette tip is connected to the connecting portion of the piston-stroke pipette, which is acted upon by the negative pressure/overpressure via a cylinder piston of the piston-stroke pipette that can be moved electrically in a hollow-cylinder-shaped piston chamber.

A dispensing tip is used with direct displacement devices in which there is essentially no air cushion between the piston and the liquid sample during pipetting. Here, the piston is part of the dispensing tip. This has a container part acting as a piston cylinder with a container mouth and a container opening in which the piston engages in order to be displaceably arranged in the piston cylinder and to be able to displace the entire aspirated liquid content in a final position. When connecting the dispensing tip to the pipetting device designed as a direct displacement device, the container part is connected to the connecting portion of the pipetting device, in particular by plugging or screwing on, and the piston couples to the moving member by means of a coupling device. Dispensing tips are in particular disposable products and are preferably made of plastic. Depending on the required maximum liquid volume, different dispensing tips are used with the direct displacer. Typical nominal volumes of commercially available dispensing tips are, for example, 100 μL, 200 μL, 500 μL, 1 mL, 2.5 mL, 5 mL, 10 mL, 25 mL, 50 mL.

The hand-held pipetting devices described in the context of the present invention are preferably, but not exclusively, hand-held computer-controlled piston stroke pipettes with electric piston drive, also referred to as hand-held electric pipetting devices or hand-held electric piston stroke pipettes. However, the pipetting devices that can be used with the system according to the invention can also be mechanical, in particular manually driven, pipetting devices. One way to realize this would be to perform an automatic identification procedure to identify a pipetting device, in particular by scanning the barcode or QR code that may be attached to the housing of a mechanical pipetting device and that contains identification data (an ID, e.g. serial number) as information. This information can then be stored in the corresponding method data set to document which pipetting device(s) was used. Recording of pipetting operations of a mechanical pipetting device can be enabled via an acquisition system, e.g. a sensor of the pipetting device or an image acquisition and processing system of the system.

The invention also relates to a method of using the system according to the invention, comprising the steps:
a) starting, in response to a user input, the recording of a first recording portion of the first method recording, wherein a first subset of the first sequence of parameter sets of the first experiment section is recorded and stored as a first method data set in the data storage device,
b) pausing, in response to a user input, the first method recording; and preferably: c) selecting, in response to a user input and the first method identification data, the stored, first method data set and terminating or continuing the first method recording recording the first experiment, wherein in the case of continuing, recording of a second recording portion of the first method recording is started in which the second subset of the first sequence of parameter sets of the second experiment section is recorded.

Further preferred embodiments of the method and system according to the invention will result from the following description of the embodiment examples in connection with the figures and their description. Identical components of the embodiments are identified by substantially the same reference signs, unless otherwise described or otherwise apparent from the context. Showing:

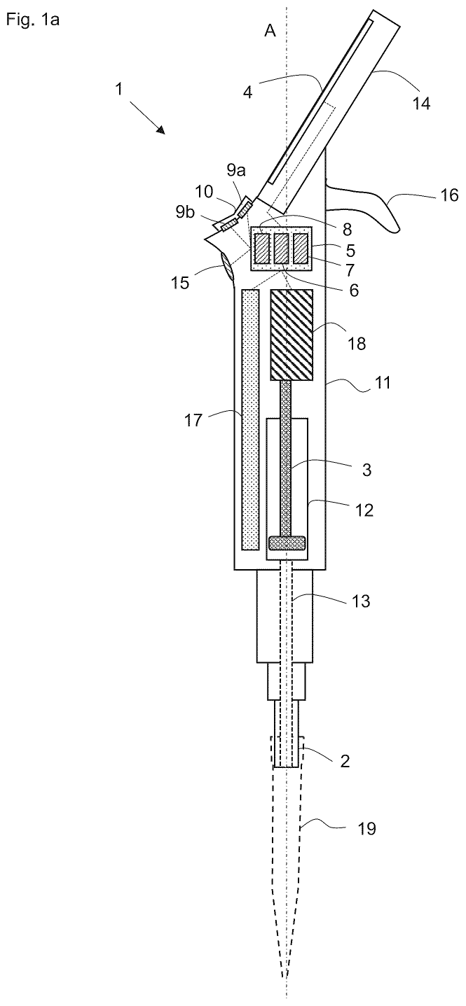

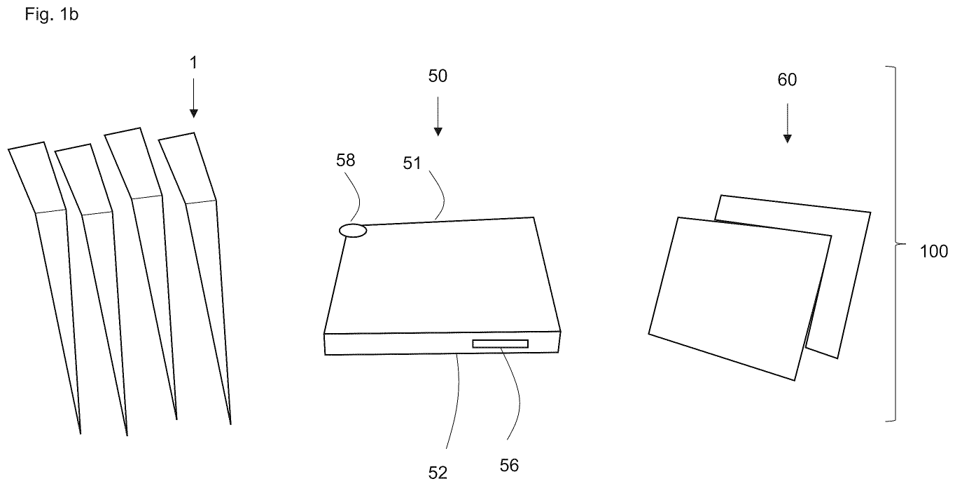

FIG. 1a illustrates a schematic side view of an embodiment of a pipetting device that can be used with a system according to the invention of FIG. 1b.

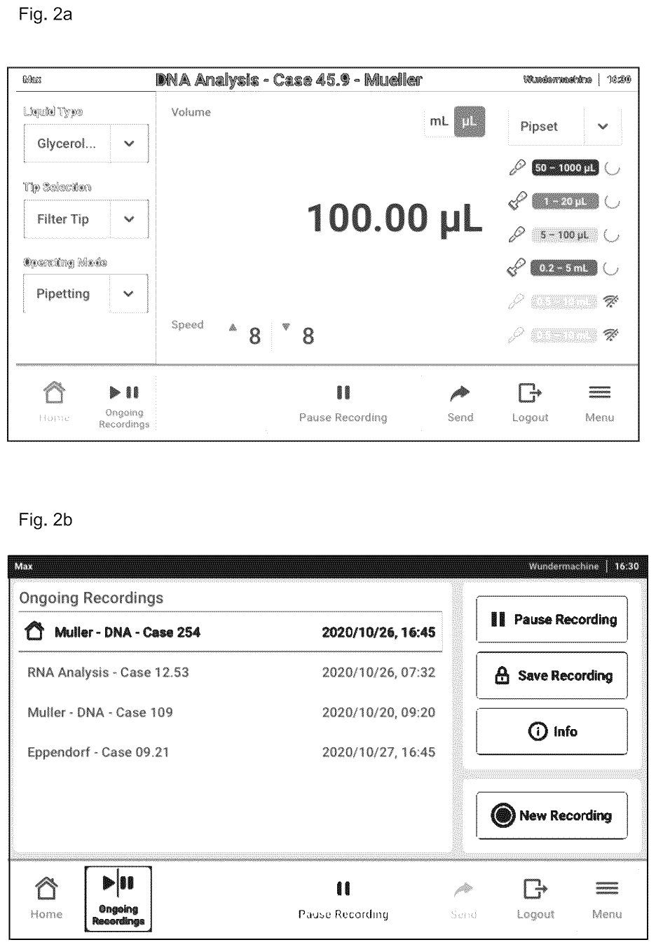

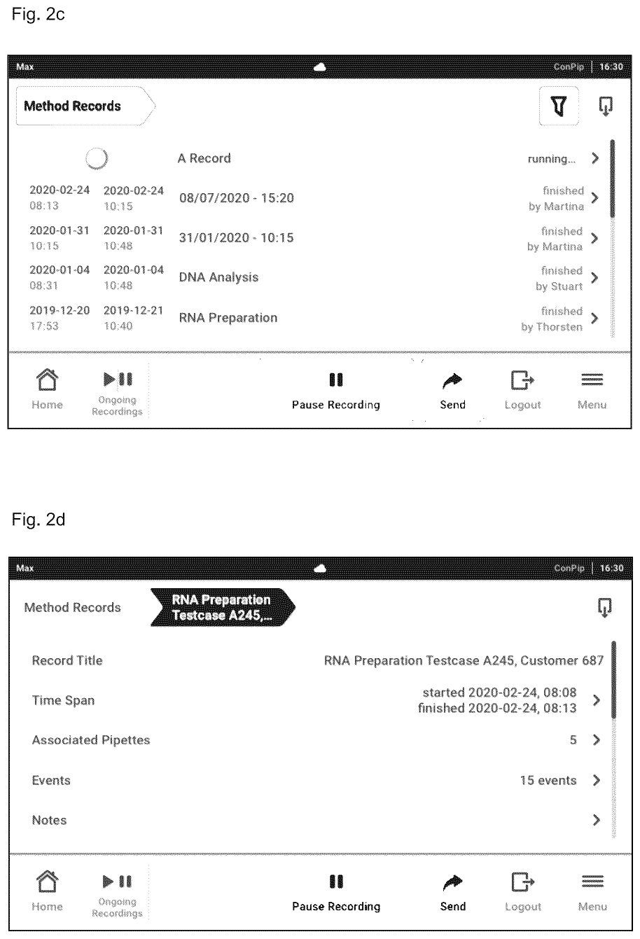

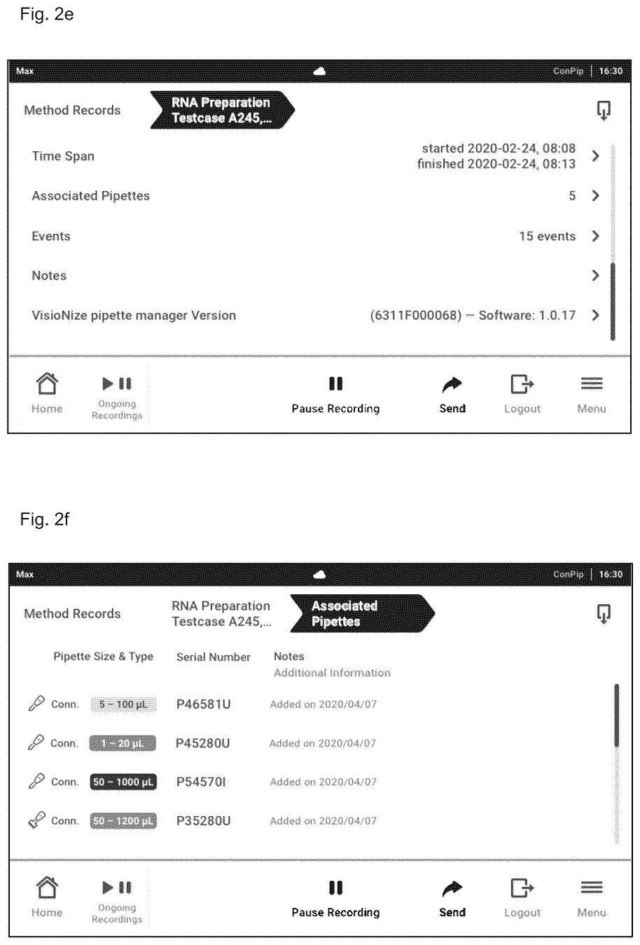

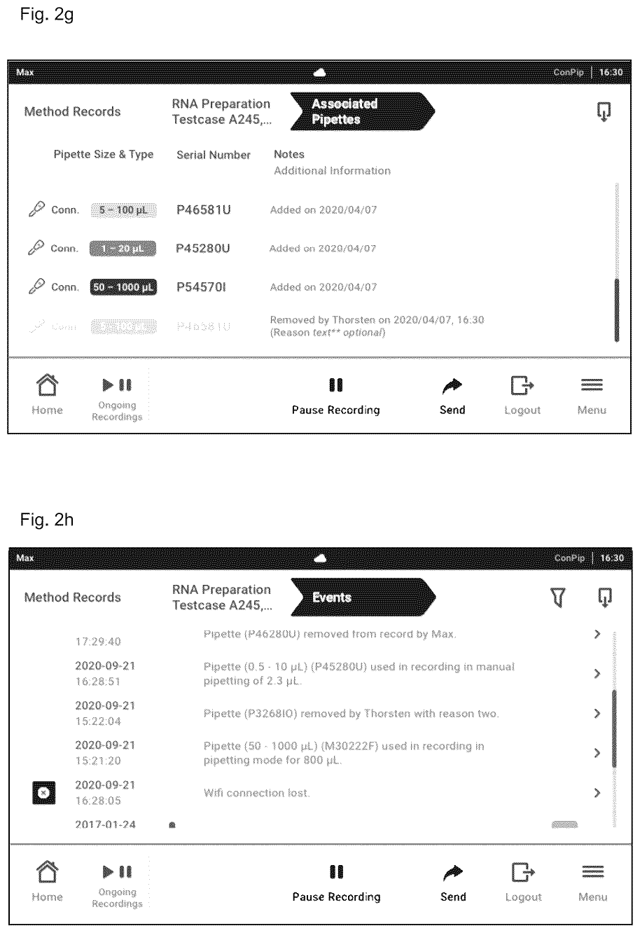

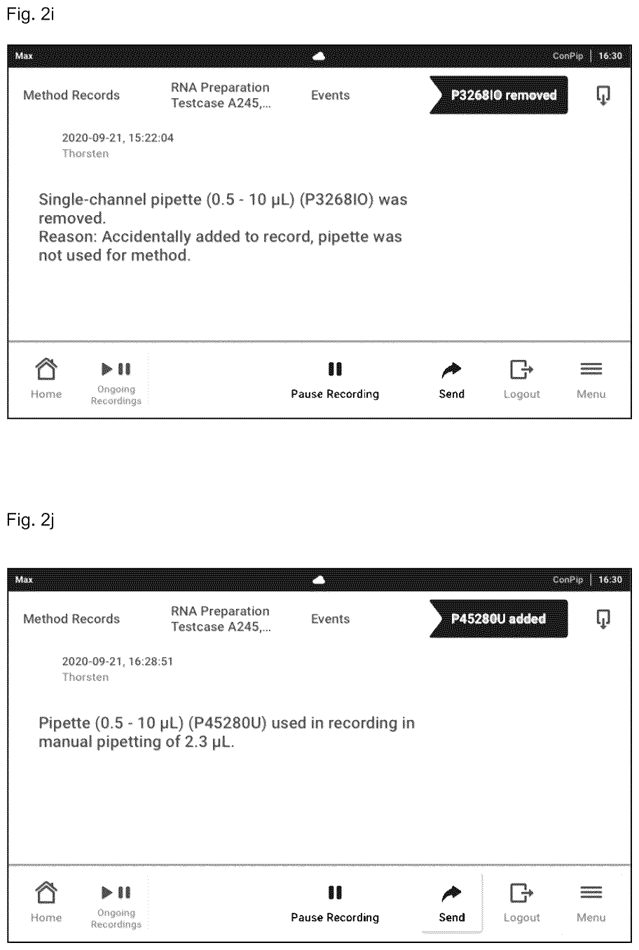

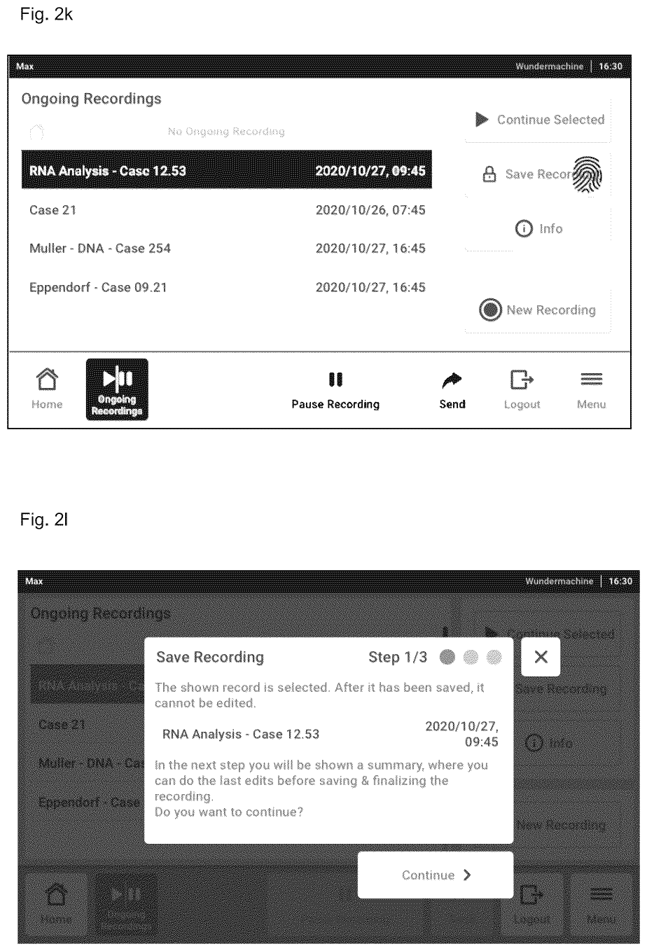

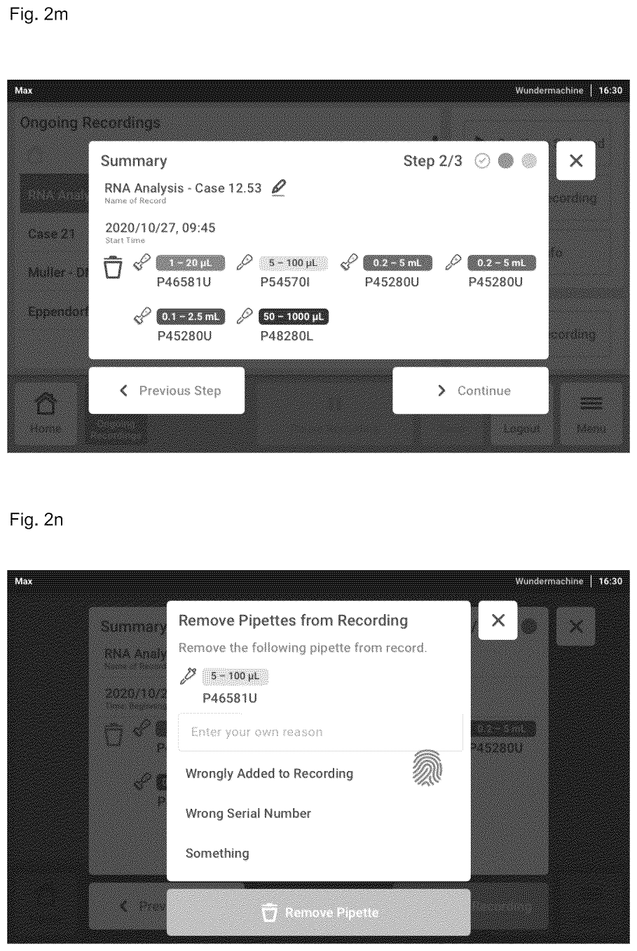

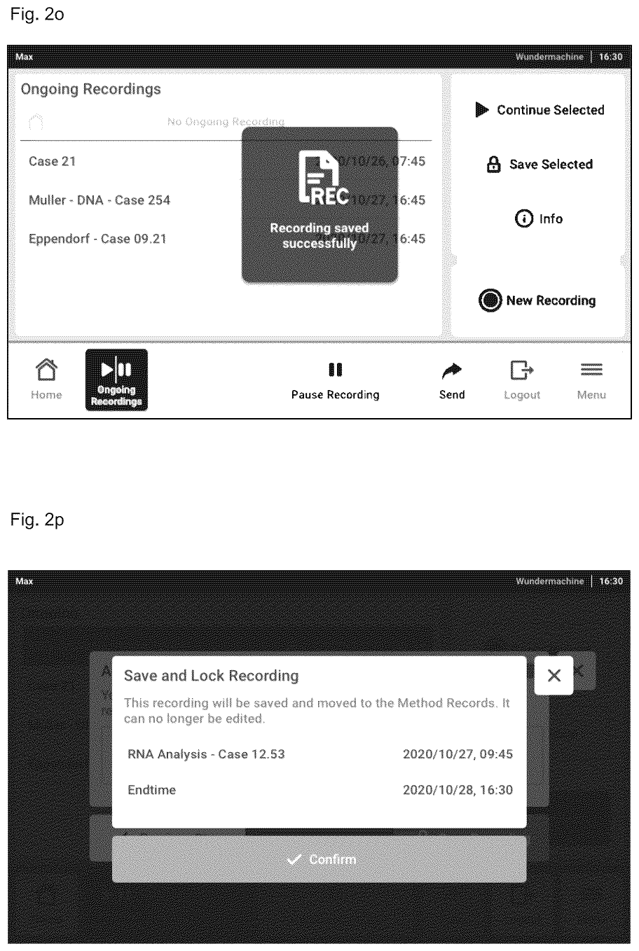

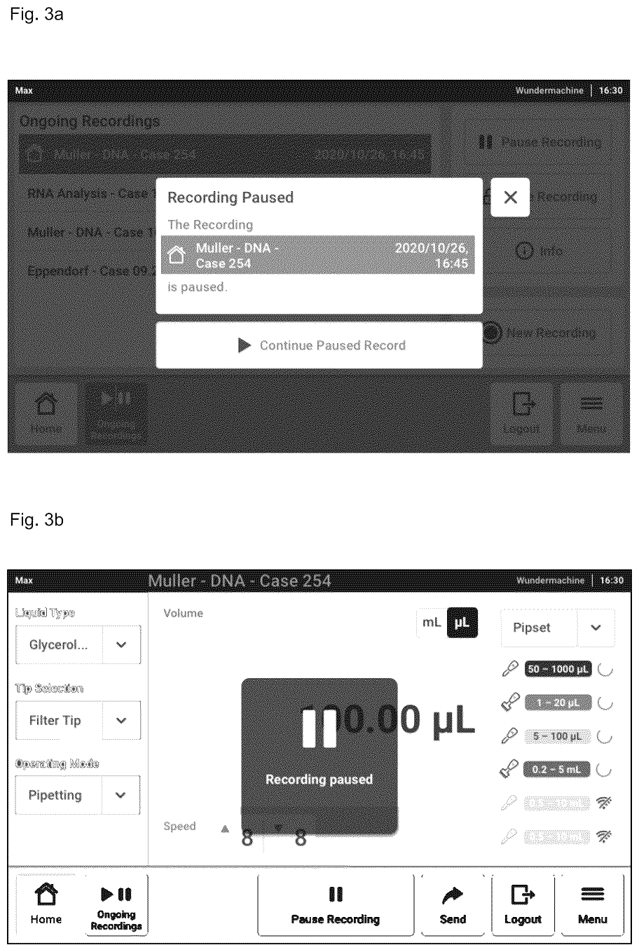

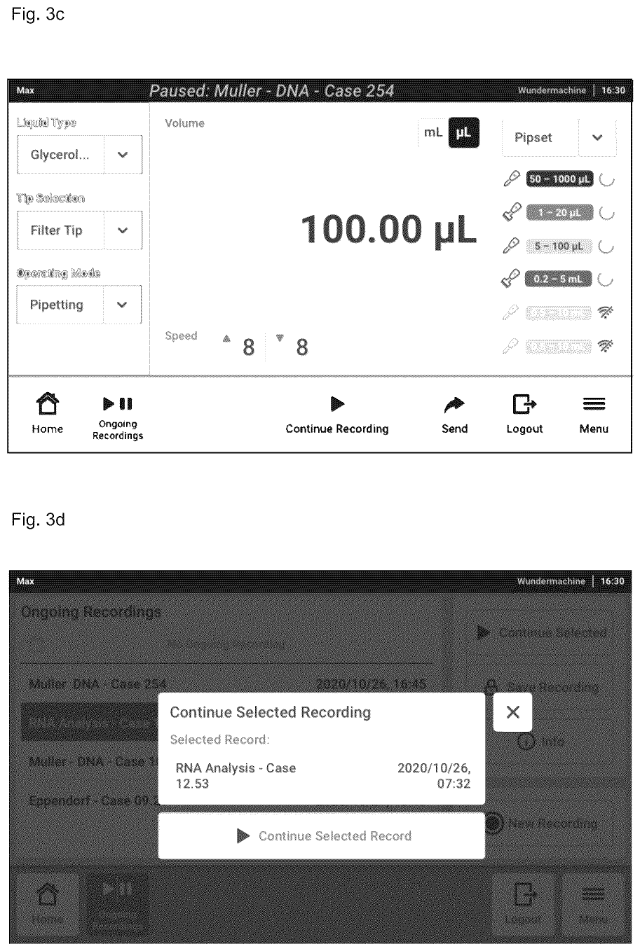

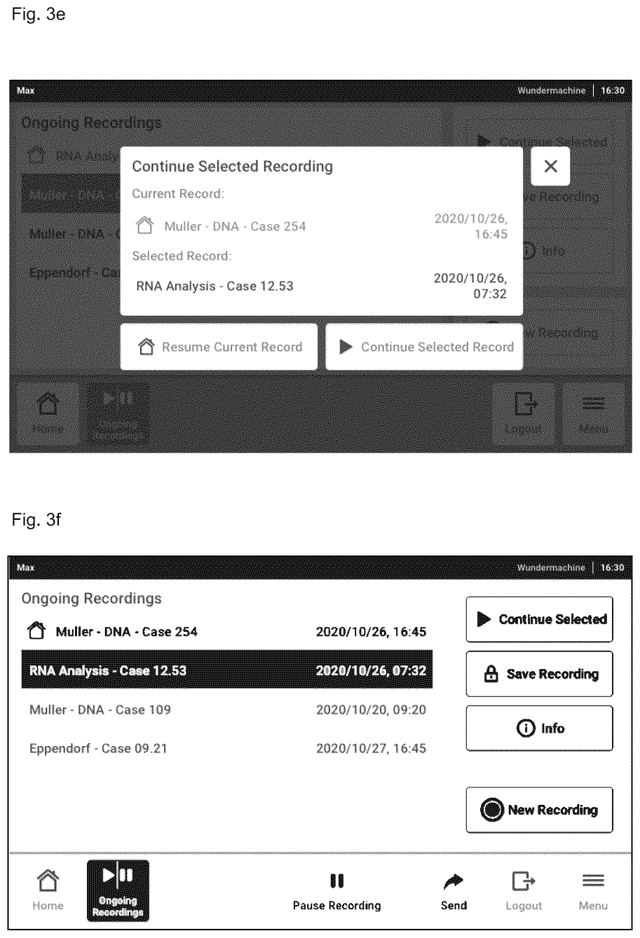

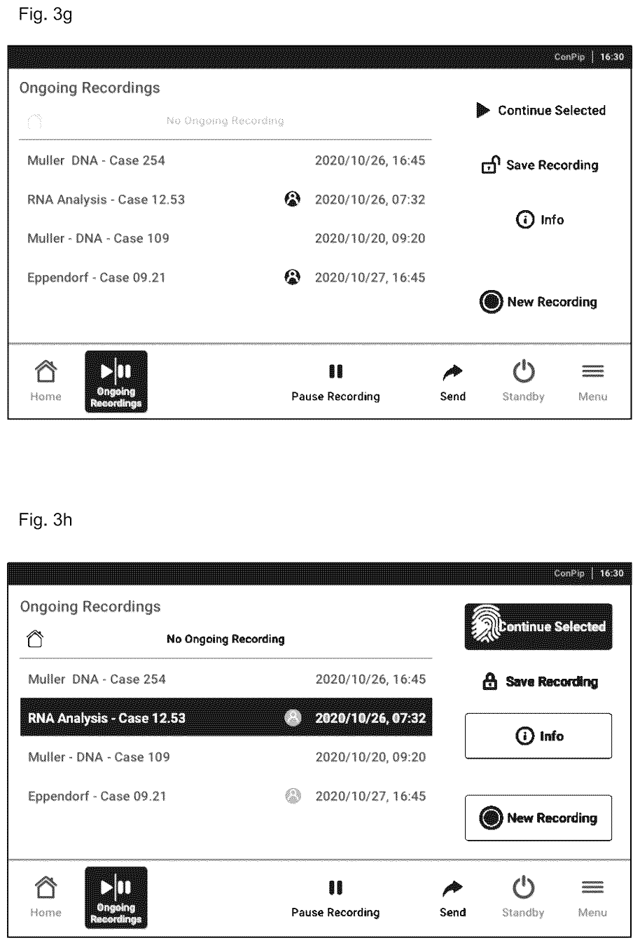

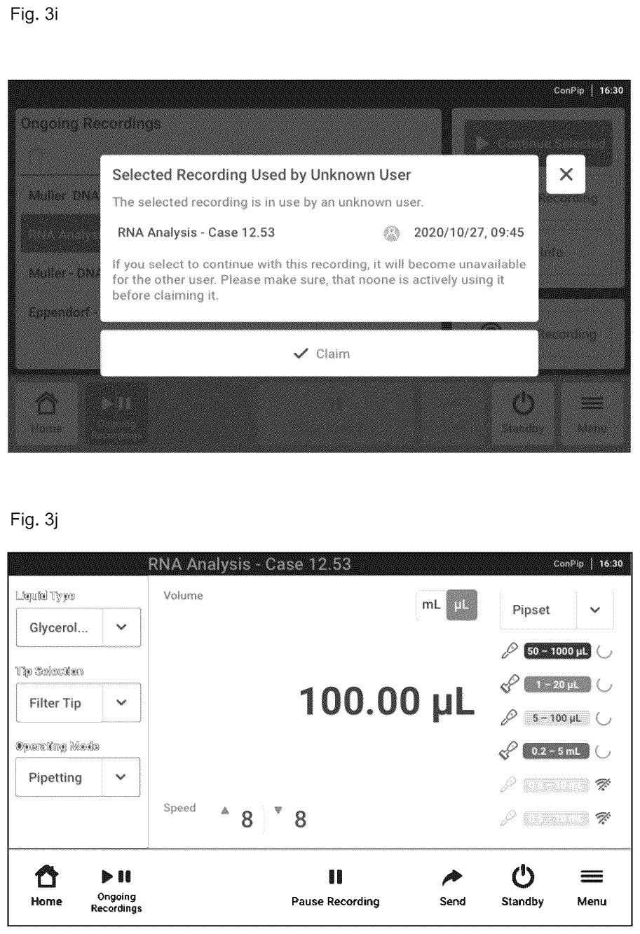

FIG. 1b illustrates a system according to the invention with which the pipetting device of FIG. 1a can be used and on whose user interface display the screen pages shown in FIGS. 2a to 2p and 3a to 3j are output as components of a GUI.

FIGS. 2a to 2p each illustrate a screen page that can be output on the portable computing device that forms part of the system according to the invention in FIG. 2.

FIGS. 3a to 3j each illustrate a screen page that can be output on the portable computing device that forms part of the system according to the invention in FIG. 2.

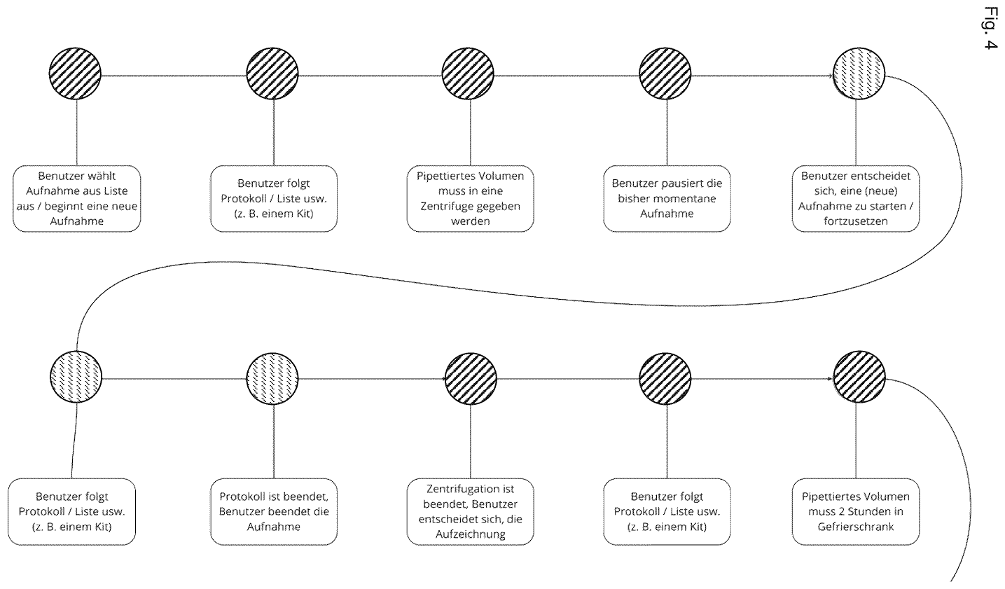

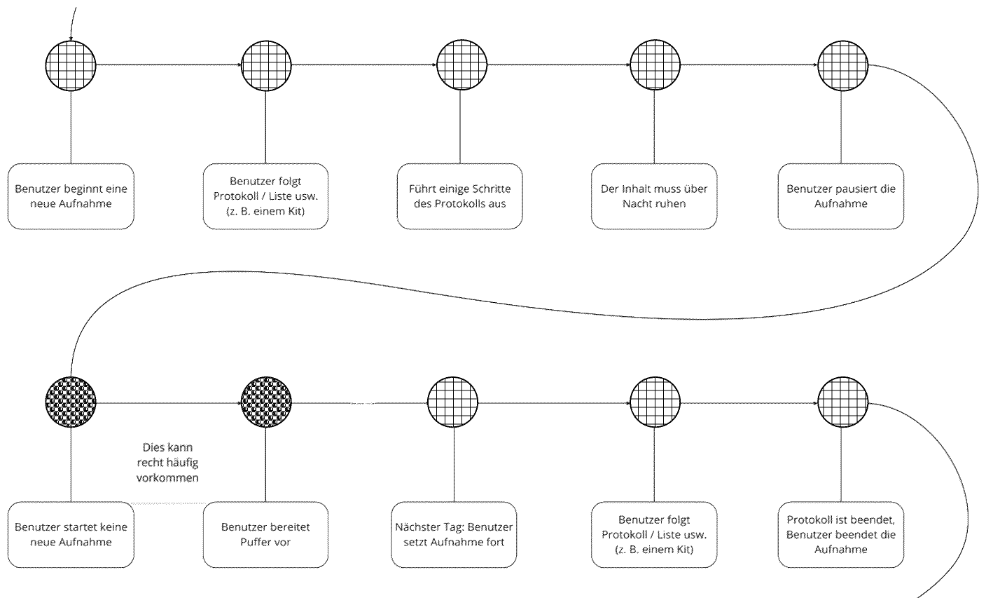

FIG. 4 illustrates the sequence of user interactions that may be performed in an application of the system according to the invention in its exemplary embodiment during one or more experiments with interleaved recordings, or that are performed in an exemplary method according to the invention.

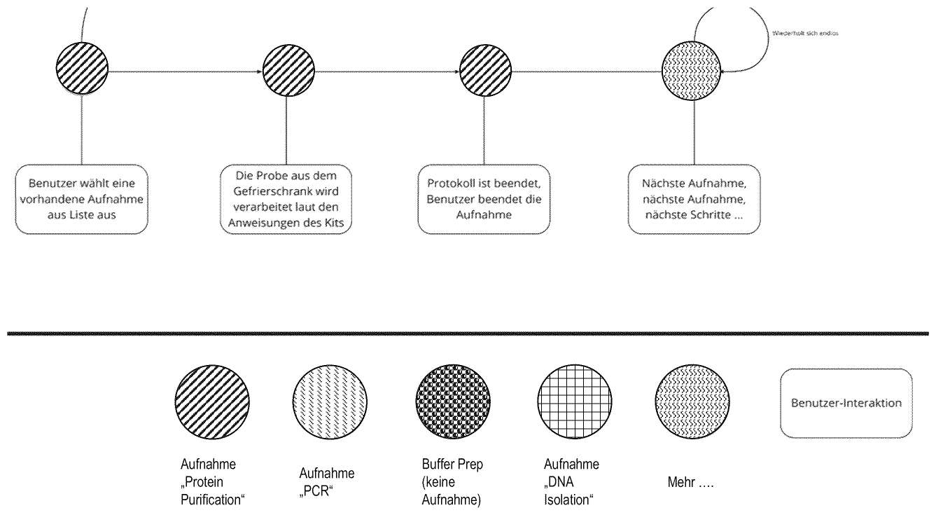

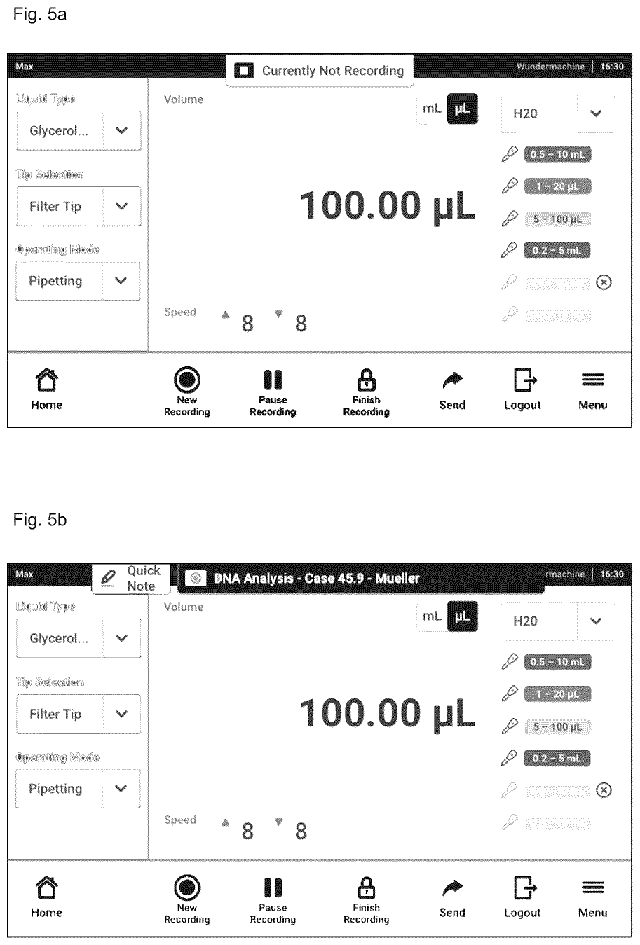

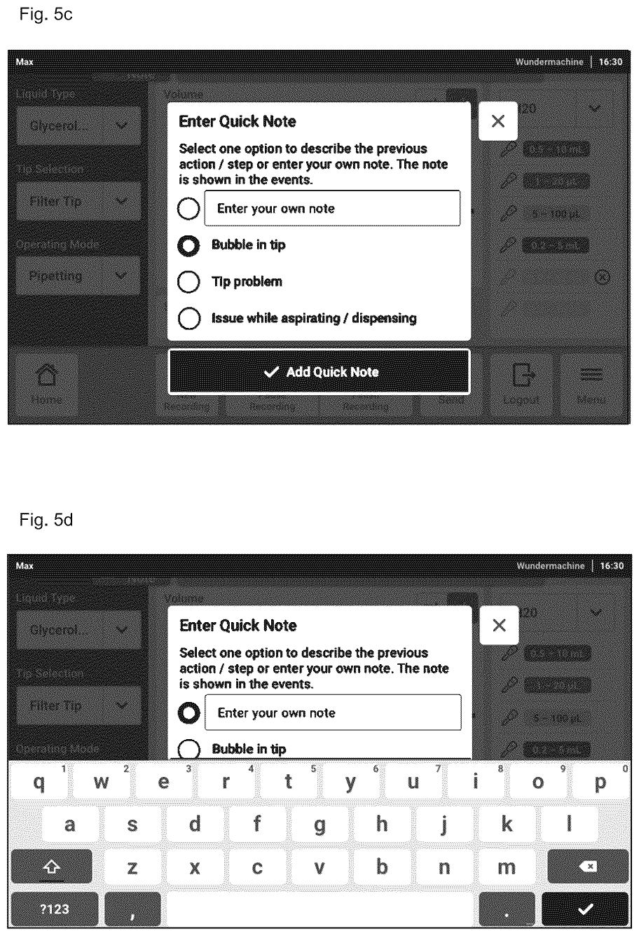

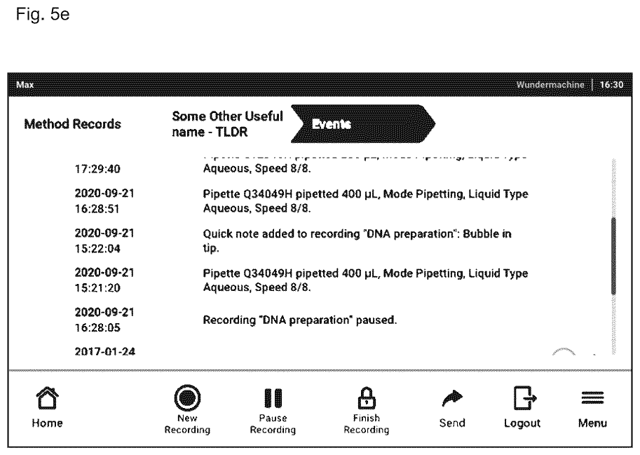

FIGS. 5a to 5e each show a screen page that can be output on the portable computing device that forms part of the system according to the invention in FIG. 2.

FIG. 1a shows a hand-held pipetting device 1 for pipetting at least one liquid sample, in this case an air cushion pipette 1, which, in addition to optionally further similar pipetting devices, can be used in the system 100 according to the invention of FIG. 1b. The pipetting device 1 is assigned a serial number which serves to uniquely identify it in the system. This identification data is stored in the data memory of the pipette and can be read by the system. This pipetting device 1 has: a connecting portion 2 (working cone) for connecting at least one pipetting container 19 from FIG. 1a, in this case a pipette tip, a moving member electrically controlled by the control device 5, in this case the piston 3—in the case of electric dispensers, the moving member is a motorized moving component of the dispenser which is connected to the piston of a dispenser syringe-, for aspirating the at least one sample into the at least one pipetting container 19, holding the sample in the at least one pipetting container and dispensing sample from the at least one pipetting container when performing a pipetting operation,

a touch-sensitive screen 4 on the front surface 14 A of the head section 14 of the pipetting device, for inputting the parameter values of user-definable pipetting parameters, wherein a parameter set of pipetting parameters completely defines the pipetting operation, wherein the electrical control device 5 comprises a data processing device 6 programmed to control the moving member 3 in dependence on the at least one pipetting parameter, and a data memory 7 and a communication device 8 for wireless communication with an external data processing device—in particular the portable computer device 50, with two operating elements (synonym: actuating elements) 9a and 9b, which can be actuated by the user by means of an operating rocker 10. By means of an actuating element, a pipetting operation in particular can be started by the user.

The communication device 8 is to be understood as a “second communication device” in the sense of the claim and is in particular set up to exchange data with the corresponding (first) communication device 58 of the portable computer device 50. In particular, configuration data is received by the pipette 1 from the portable computing device in order to operate the pipette 1 in an operating mode selected at the computing device 50, in particular with the pipetting parameters which the pipette also receives from the computing device 50, where the pipetting parameters have been previously set by the user. In particular, the exchanged data may include information about: an operating mode set on the pipette 1, the pipetting parameters applied during the pipetting operation, start and/or end time of the pipetting operations or their sub-steps, identification data—e.g. serial number—of the individual pipetting devices used, identification data of the—optionally previously authenticated or selected—user, who will usually be the executive in charge of an experiment, and other data that may be provided by other devices and/or sensors of a laboratory involved in the experiment. These data can then be stored as data of the method data set.

The pipetting device comprises an electric motor 18 which is powered by an accumulator 17 and which moves the piston rod or piston 3 within the cylinder piston 12 so that an aspiration pressure or a dispensing pressure is the result in the pipetting channel 13. Precise dispensing can be achieved by means of the electronic control system. An ejection button 15 is provided to allow ejection of the pipette tip 19 from the connecting portion 2 by means of an ejection sleeve of the pipetting device. The data processing device is programmed in particular to abort an ongoing pipetting operation in the event of actuation of the ejection button 15.

Also shown in FIG. 1b are further external devices 60, representative of tablet PCs or smartphones, which can optionally be used to set or read out the parameters set on the computer device 50, in particular pipetting parameters.

The selection of pipetting parameters of a parameter set defining the desired semi-automated pipetting operation and the setting of the values of these pipetting parameters is performed by means of a GUI on the computer device 50, as shown in FIG. 1b. The computer device 50 has a housing 51 in which a touch screen 52 is integrated. This serves as the user interface device 52 of the system. The data processing device 56 of the computer device 50 is programmed to control the method recording(s) of the system. The data processing device 56 of the computer device 50 is programmed to display in the touch screen 52 screen pages of the GUI by means of which the system is controlled by the user. The corresponding control software is a modified version of the “VisioNize® pipette manager” control software. Such screen pages are shown in FIGS. 2a to 2p and 3a to 3j. The logging function implemented with this software by means of pausable, in particular nestable, method recording is explained below.

Within Method Recordings, the [Process/Run] Records function is extended for the VisioNize® pipette manager. From an Ongoing Recordings screen, the user can start a recording (also referred to as a recording) to create a time frame that defines when the recording will occur, as well as start a new recording, edit paused recordings, or save a recording from a special page accessible from the bottom bar.

The recordings can be exported and result in a PDF according to the set filters.

A method recording is (unless an exception occurs) a single user scenario.

In a home screen of the GUI (shown in FIG. 2a), the operating mode of a pipette 1 connected and assigned to a method—identifiable via method identification data- and associated pipetting parameters can be set, transmitted to the pipette 1 (or even several pipettes 1 of the same type or other pipettes) and thereby configured.

The method recording is available as a function to pause a recording on the Home screen, from the Ongoing Recordings screen to resume/pause/save/start a new recording, and from the Menu to access the function. The “Menu” screen shows the “Method Records” button. When touched, the user has access to all saved and completed records.

At the beginning of the lab work, a user may decide to document the subsequent interactions. This is especially beneficial to ensure that an audit that may occur at a later time can be successfully passed. The method recording feature supports this requirement. The main interactions are navigated from the home screen.

On the start screen shown in FIG. 2a, the information whether a recording is running is displayed in the status bar. One of three statuses can be displayed in the status bar:
Record
No record
Paused

The bottom bar has a button associated with the method recording. It shows “Ongoing Recordings” with a play/pause icon. It behaves like the menu in the bottom bar.

The “Ongoing Recordings”, see screen in FIG. 2b, can be accessed via the “Ongoing Recordings” button in the bottom bar. The topmost list entry is the one currently being recorded. It is highlighted and shows a house icon—in reference to “in use” on the home screen. The date at the end of the list item shows the date it was created (year/month/date, hr:min). The list items are separated by a separator line.

The list is dynamic and uses only as much space as needed—if more recordings are running than can fit in one screen, a scroll bar is displayed. The buttons on the right side allow the user to interact with the recordings. The buttons are disabled as long as no method set is selected from the left list. “New Recording” is always enabled and starts a new recording. The first button is context sensitive. It can mean either “Pause Recording” or “Continue Selected”: The “Pause Recording” button is only displayed when the active recording is highlighted. Pressing it stops (pauses) the recording. The “Continue Recording” button is only displayed if the active recording is highlighted.

is displayed and enabled when a recording other than the topmost recording is selected (it is disabled when no recording is selected). “Save Recording” allows the user to save the recording. After that, it cannot be edited. “Info” shows the user information about the selected recording. Here the user can also edit the information (e.g. title).

FIG. 2c: For each method recording (method data set) a start and end date (with date & time) is displayed. The title of the record is displayed as well as the status of the record. The status of a recording can be one of the following:
“running” . . . →indicates that a recording is in progress (multiple recordings can be displayed as “running”, e.g. when multiple users are recording via different devices—and later multiple recordings can be running when a user switches between different recordings by pausing a recording and continuing another recording via “continue” or starting a new recording, and while a recording is running, the activity indicator is displayed.
“finished” (“completed”)→a recording that has been finished and then locked so that it can no longer be edited.

One of the following information elements can be displayed below the status:
“by user” (“from USER”)→with the name of the user or (if user management is deactivated) of “anonymous user”.

Multiple recordings can be displayed in this list if user management is disabled and multiple sessions are active for the “Unknown User” with multiple recordings running.
if the user management is enabled, only the current one will be displayed by the logged in user on this device
if the user management is deactivated, after recordings have been made with the user management activated, the name of the user is displayed here are displayed.

FIG. 2d, 2e: Clicking on the drill-down indicator next to a recording takes the user to an overview of the recording, which displays the following information:
Name of the record in the breadcrumb bar (“active” part of the breadcrumb in blue background color)
Recording title (as header) and the recording title
Time span (as header) with the start & end time of the recording
Associated pipettes (headers) with a number
   All pipettes used for the acquisition (if the pipette was actively used by the user) and a drill-down indicator
   only the pipettes that are still part of the recording are added for this number (pipettes that have been removed from the recording are excluded)
   Removed pipettes are displayed in gray at the bottom of the list (with—if given—a reason for removing the pipette from the record)
Events(header) and the number of events with a drill-down indicator (the number increases as more events occur while a recording is in progress).
Notes (optional→only if a note was added when saving the recording).
VisioNize® pipette manager version (header) with the installed software version at the time of recording

FIG. 2f, 2g: The breadcrumb bar also shows the current level reached by the drilldown interaction on the previous screen. Clicking on the “RNA Preparation Testcase A245” button opens the page at the next higher hierarchical level. At the top are also the options:
Export

The associated “Associated Pipettes” page shows a list of all pipettes that were actively used during the acquisition. The page is structured by a header with the following elements:
Pipette size and type
Serial number
Notes (with “subtitle” information”)
   Additional information

And a list of the following items:
Icon for pipette (Indicates single or multichannel pipette type)
Volume badge
Serial number of the pipette
Additional information that may consist of:
   “Added on” (“Added on”) DATE”
   “Removed by” USER on DATE+time (opt. text with reason)” (removed pipettes are the last in the list).

The export triggered in this screen will be exported as a PDF to a USB stick:
the associated pipettes listed here (with all information displayed) as well as all deleted pipettes (marked accordingly), the recording names, the user, and the date and time of recording

The removed pipettes are displayed in gray. The volume badge is also displayed in a deactivated state.

FIG. 2h, 2i, 2j: The events are grouped around a method record and are part of a method data set. The event detail should show what was “done”, specifically whether a pipette was added or removed from the record. It should also indicate the chosen reason for removing the pipette and the date and time when this occurred. The user who triggered this action should also be indicated here (especially when removing a pipette).

FIG. 2k, 2l, 2m, 2n, 2o, 2p: The user can change in the screen with the running recordings select the “Save Recording” option. This is always enabled as long as a recording is selected from the list. A three-step wizard is displayed. The name of the recording and the start time are displayed to the user in the dialog. Once a recording is saved, it can no longer be edited—it is “locked”. The overlay that appears includes the following elements:
a title,
a pedometer for three steps (in accordance with the dialog (wizard))
a short explanatory text,
the name of the recording (with date),
a “Next” button,
a “Close” button (X).

(The fingerprint displayed on the screen page, if any, symbolizes a touch of the corresponding button by the user).

The user can select which pipettes are to be removed by tapping on the pipette. The pipettes are highlighted as selected. Once (at least one) pipette is selected, the user can tap on the dropdown box and an overlay is displayed. The dropdown box is otherwise displayed as disabled in

gray. In step 2/3, the user is shown a summary of the selected exposure. The summary shows:
Title (under which the recording is saved) with an editing option on the screen
Time of recording (start)
Associated pipettes; a scroll bar is displayed if too many pipettes are listed here; the associated pipettes are displayed as a list, four pipettes side by side, and list the pipette symbol, badge, serial number with a delete option

The information of the pipettes are:
Icon of pipette by type (single channel, multichannel, mechanical)
Volume badge
Serial number

The user can change the title (on the screen) or remove an eyedropper. The delete key enables the following:
a dropdown box is displayed below the pipettes
the edit icon turns blue
the user can select pipettes to be removed from the list by tapping on them

In step 2/3, the user can edit the title of the recording (before saving) and delete the associated pipettes that are displayed. When deleting the pipettes, a reason can (but does not have to) be selected as to why the pipette was removed from the recording. If a reason is specified, the reason will be displayed in the export and recording details. The user can also specify a personal reason. Once a pipette has been removed, it is no longer displayed in the overview in step 2/3. Multiple pipettes can be removed at once. After the user has edited something, the “Warning” pop-up dialog is displayed to the user when the Close Wizard is selected. This is to prevent accidental data loss. As step 3/3, the user can optionally enter a note about the recording. When “Save Recording” is selected, an overlay is displayed, this once again informs the user that the recording will be saved and then locked and cannot be edited. If this is accepted by the user, an overlay will appear in the Recordings In Progress screen informing the user that the recording has been successfully saved. In the list displayed here, the recording is now missing as it has been moved and can now be found in the Method Recordings (accessed via the menu). The reason, time & date, as well as the user and which pipette (serial number, type, volume) why a pipette was removed from the recording will be saved and listed in any report created from that recording. The information is displayed to the “Associated pipettes” overview but the pipette is no longer counted to the “Associated pipettes” (Numeric in the overview list). There is no way to permanently delete a pipette from a recording—it is just an omission from the list stored within the VisioNize Pipette Manager software. In the third step it is possible to add a note to the recording. The text input field enlarges while entering the information.

FIG. 3a, 3b, 3c: The user can pause a recording. There are two different scenarios and procedures in the GUI. When the user is in the recording in progress screen and selects “Pause Recording”, an overlay is displayed giving the user several options. The user can choose to “Continue Paused Record”, which will continue the recording; and “unpause”. On the other hand, the user can close the overlay with the “Close” button. Closing the overlay deselects the recording and displays “No ongoing recording” as the top entry in the ongoing recordings screen. The other option is found on the homescreen—here the user can pause recording by tapping the button in the bottom bar. When selected, an information panel will pop up informing the user that the recording is paused. The status bar will still show the recording, but with the words “Paused: Title”. The button in the bottom bar now reads “Continue Selected” with a play icon. This allows the user to easily pause and resume recording from the Home screen.

FIG. 3d, 3e, 3f: When a recording is displayed as part of the list of recordings in progress, the user has the option to select a recording to continue. When a recording is selected, an overlay is displayed and depending on whether-or-not a recording is currently in the homescreen, the overlay is displayed in one of two states.

If there is no recording in the “Homescreen slot”, the “Resume Selected” overlay shows only the newly selected recording and the button group in the overlay shows only one button. The user has the option of resuming the selected recording or

close the overlay via the close button. When the overlay is closed, the recording is still selected but not continued. When clicking “Continue Selected”, the user is brought to the home screen where the continued recording is running, and all—now following—interactions are linked to the recording.

If there is a recording in the homescreen slot, the overlay has a two-button group at the bottom and shows both recordings in the overlay. The homescreen

Recording shows the Home icon and is highlighted in blue—this is to easily distinguish the two and to reuse the same color and icon usage as shown in the recording in progress screen. If the user selects the “Resume Current Record” option, recording will resume from the Home screen slot. If “Continue Selected Record” is selected, the newly selected recording will be resumed. In both cases, the user is brought to the homescreen and the name of the recording is displayed in the status bar. When closing the overlay with the close button, the overlay is closed and the selected record is still selected.

FIG. 3g, 3h, 3i, 3j: When user management is disabled, “sessions” are used to correctly identify “different” user sessions. The user will see all currently running or paused recordings on the “Running recordings” page. The recordings that are active within that session on that device are displayed without any indication that another “user” is connected to them. A user can claim another recording to continue working on it. The Recordings In Progress page and the listed recordings indicate whether a recording is associated with another session via the user indicator. A user can “claim” the recording by selecting it (it will then be highlighted) and choosing “Continue Selection”. A popup is then displayed informing the user that this recording is currently linked to another session. The name of the recording, the start type, and the user icon are displayed. Clicking “Claim” makes the recording an active recording for that session, moves it to the homescreen slot, and takes the user to the homescreen where the claimed recording is running.

Via a social log, the user is asked to verify that no one else is using this record, as there is no way to ensure that the record is actually free. A record cannot be merged at a later time, so it is important that users communicate and are informed about this.

FIG. 4 shows the sequence of user interactions (BI) that may be performed in an application of the system according to the invention in its exemplary embodiment during one or more experiments with interleaved recordings, or that are performed in an exemplary method according to the invention:
1: User selects recording from list/starts a new recording
2: User follows protocol/list etc. (e.g. a kit)
3: Pipetted volume must be placed in a centrifuge
4: User pauses the recording that is currently in progress
5: User decides to start/continue a (new) recording.
6: User follows protocol/list etc. (e.g. a kit)
7: Protocol is finished, user stops recording
8: Centrifugation is finished, user decides to stop recording
9: User follows protocol/list etc. (e.g. a kit)
10: Pipetted volume must be in freezer for 2 hours
11: User starts a new recording (until step 12: This can happen quite often)
12: User prepares buffer
13: Next day: User continues recording
14: Protocol/List etc. (e.g. a kit)
15: Protocol is finished, user stops recording
16: User selects an existing recording from the list
17: The sample from the freezer is processed according to the instructions of the kit
18: Protocol is finished, user stops recording
19: Next shot, next shot, next steps . . . .
Loop at step 19: Repeats endlessly

Color legend for FIG. 4:
turquoise: Recording (record=recordin”) “Protein Purification
violet: recordi″g “PCR”
orange: Buffer Prep (no recording)
green: Recordi″g “DNA Isolation”

The method records performed by the system 100 use method identification data, particularly individual method identification data, to distinguish the different methods, particularly the first and second methods and their respective method records. For example, in FIG. 2b, first method identification data is associated with the method designated therein “as “Muller—DNA—Case “254”, second method identification data is associated with the method designated “as “RNA Analysis—Case 12.53”, third method identification data is associated with the method designated as “Muller—DNA—Case “109”, and fourth method identification data is associated with the method designated as “Eppendorf—Case 09.21”. The first, second, third and fourth method identification data—and any other method identification data to be used—are clearly distinguishable from each other. In particular, they are suitable to be able to uniquely select one of the correspondingly existing first, second, third or fourth method records, and/or to uniquely continue or terminate the method record. The content of the method identification data is not important, for example the digits 1, 2, 3, 4—or also their respective binary representation—would be suitable to form the respective method identification data.

The data processing device 56 of the computing device 50 is programmed to control the method record(s) of the system, in particular the aforementioned method records of FIG. 2b. In particular, the data processing device 56 of the computer apparatus 50 is programmed to,
by means of the first communication device 58, after the execution of at least one pipetting operation associated with a first method, to receive from the at least one second communication device 8 recording data containing the parameter values on the basis of which the at least one pipetting operation was configured and executed by at least one pipetting device,
by storing the recording data as a first method data set in a data storage device—here of the portable computer 50, in particular as a component of the data processing device 56—a first method recording is performed, to which first method identification data are assigned,
pause the execution of the first method recording in response to a user input occurring at the user interface device, and
continue the paused execution of the first method recording, i.e. “the paused first method recording” for short, as a function of a user input at the user interface device and in particular as a function of the first method identification data, or to end the paused first method recording as a function of a user input at the user interface device and in particular as a function of the first method identification data. In particular, termination is performed here by the user touching an input field described as “Save Recording” (see FIG. 2l) of the touchscreen. In doing so, the data processing device 56 detects that—here in the example—the first method identifiable by first method identification data (here with the designation “Muller—DNA—Case 254”) is meant. Continuation of this method or method recording is then preferably no longer possible after storage (FIG. 2p). The continuation, in particular of the first method recording, takes place by means of the GUI optionally in such a way, as is shown on the basis FIG. 3a, 3b, 3c. Here, too, the data processing device 56 detects that—here in the example—the first method identifiable by first method identification data (here with the designation “Muller—DNA—Case 254”) is meant.

In this regard, the data processing device 56 is also programmed to continue the first method recording—e.g., “Muller—DNA—Case 254”—by
in response to the user input and the first method identification data, the first method data set stored during a first recording portion of the first method data set is selected and the recording of a second recording portion of the first method recording is started.

In this regard, the data processing device 56 is also programmed to,
after pausing the first method recording prior to starting recording of the second recording portion of the first method recording, perform a second method recording to which second method identification data different from the first method identification data is assigned;

In particular, this interleaved recording of two methods is accomplished by the data processing device 56 being programmed to,
receive, by means of the first communication device 58, after the execution of at least one pipetting operation associated with a second method, from the at least one second communication device 8 recording data containing parameter values on the basis of which the at least one pipetting operation of the second method was configured and executed,
perform the second method recording by storing this recording data as a second method data set in the data storage device, the second method data set containing in particular second method identification data,
pause the execution of the second method recording in response to a user input occurring at the user interface device, and
continue the paused, second method recording in dependence on a user input at the user interface device and in particular in dependence on the second method identification data, or end the paused, second method recording in dependence on a user input at the user interface device and in particular in dependence on the second method identification data.

FIGS. 5a to 5e each show a screen page that can be output on the portable computing device that forms part of the system according to the invention in FIG. 2.

FIG. 5a shows a screen page that forms the “homescreeen” of a logged-in user, in the case that currently no method recording is taking place. This is signaled to the user by displaying the information field marked with the text “Currently Not Recording” at the top of the screen page.

FIG. 5b shows a screen page that forms the “homescreeen” of a logged-in user, in the case that a method recording is currently taking place. This is signaled to the user by displaying the information field with the text “DNA Analysis—Case 45.9—Mueller” at the top of the screen page. The text also forms the designation of the corresponding method recording.

Referring to FIGS. 5a through 5e, the data processing device 56 is programmed to capture one or more note inputs made by the user via the user interface device during the performance of a method recording, and to store the note inputs as part(s) of the corresponding method data set with that method data set. In the home screen of FIG. 5b, a control panel of the touch screen is displayed at the top of the screen for this purpose, which is labeled “Quick Note”. Touching the control panel by the user opens an overlay window that displays a selection list of predefined note texts, here concerning possible error conditions (“bubble in tip” etc.), see FIG. 5c, each of which can be selected individually by the user and which are then saved as note inputs in the method data set. Selecting the input field for free text input (“own vote”) opens a virtual keyboard as an individual text input option (FIG. 5d).

The note input includes a text entered individually by the user, in particular by the virtual keyboard—or alternatively by a (hardware) keyboard actually assigned to the, in particular portable, computing device (not shown). Note input here also includes the option that a predefined selection list of possible note texts is provided to the user on the screen, from which the user can then select the appropriate note text. The content of such note texts here relates to a technical error observed by the user during the pipetting operation, but may also describe other typical situations, e.g., an environmental factor such as a temperature, humidity, or sample condition of the sample(s) pipetted in the method, or some other undesired or desired factor that could affect the results of the experiment in particular.

The one or more note inputs are stored here with a time stamp, which in particular enables a subsequent assignment of the one or more note inputs to other substeps of the pipetting operations of the method or generally, to another recorded event of the method recording and/or enables the chronological classification of the note. The timestamp preferably includes the time at which the “Quick Note” input field (see FIG. 5b) of the touchscreen is touched by the user and/or includes the time at which the text input option associated with the note input, in this case the overlay window in FIG. 5c or 5d, is closed.

The data processing device 56 is programmed to output the event history, also referred to as “event log”, of a method data set, i.e. the sequence of events forming the method record, on a screen—in particular of the user interface device-, in particular to output it as a list or sequence of these events, preferably with each event being represented by a corresponding descriptive text and/or a graphical symbol. The event log can be used as a protocol of the performed experiment or method recording. This is shown in FIG. 5e, in which events of the method, designated as method recording “Some Other Useful name—TLDR”, are displayed, each provided with a time stamp, as a chronological list stepping back in time from top to bottom. The note input “Bubble in tip” made according to FIG. 2c and stored with the method data set is included with the timestamp entry “2020-09-21, 15:22:04”. By the possibility of the comfortable storage of notes a still further improved method is found, in order to improve the reproducibility of experiments and/or executed methods. In this way, a logging automatically performed by the system can be refined by information individually introduced by the user.

1. A system for recording pipetting operations in user-defined experiments in which at least one hand-held usable pipetting device is used for pipetting at least one sample, wherein a parameter set of pipetting parameters defines a programmable pipetting operation of the pipetting device, and wherein an experiment includes a method defined by a sequence of pipetting operations,
where the system includes:
a user interface device with a display, in particular a touch screen,
a data processing device,
a data storage device,
a first communication device for wireless data exchange between the data processing device and a second communication device of at least one pipetting device,
wherein the data processing device is programmed to,
receive, by means of the first communication device, after the execution of at least one pipetting operation associated with a first method, from the at least one second communication device recording data containing the parameter values on the basis of which the at least one pipetting operation was configured and executed by at least one pipetting device,
perform, by storing the recording data as a first method data set in the data storage device, a first method recording to which first method identification data is assigned,
pause the execution of the first method recording in response to a user input occurring at the user interface device, and
continue the paused, first method recording in response to a user input occurring at the user interface device, or terminate the paused, first method recording in response to a user input occurring at the user interface device.

2. The system according to claim 1, wherein the data processing device is programmed to continue the first method recording by
depending on the user input and the first method identification data, the first method data set stored during a first recording portion of the first method recording is selected and the recording of a second recording portion of the first method recording is started, in particular
by receiving, by means of the first communication device, after the execution of at least one further pipetting operation, further recording data from the at least one second communication device which contain the parameter values on the basis of which the at least one further pipetting operation was configured and executed, and by continuing the first method recording by means of adding the further recording data to the first method data set and storing them in the data storage device.

3. The system according to claim 1, which comprises:
at least one hand-held pipetting device for executing at least one pipetting operation defined by the parameter values of at least one parameter set of pipetting parameters, comprising a second communication device for wireless data exchange between the hand-held pipetting device and the data processing device, wherein
the at least one pipetting device in particular comprises:
a connecting portion for connecting at least one pipetting container;
an electrically controlled moving member for aspirating at least one sample into the at least one pipetting container, holding the sample in the at least one pipetting container, and dispensing sample from the at least one pipetting container when performing a pipetting operation; and
an electrical control device which is set up to control the moving member as a function of at least one pipetting parameter,
and/or
the system includes a computing device, in particular a portable computing device, which comprises:
this user interface device,
this data processing device,
this first communication device, and
optional: this data storage device.

4. The system according to claim 1, wherein the data processing device is programmed to,
after the start of and during the pause in the recording of the first method recording, detect by means of the user interface device at least one further parameter set of at least one further pipetting operation which does not form part of the first method, and wherein the at least one pipetting device, in particular its control device, is set up to control at least one pipetting operation, in particular a moving member of the pipetting device, as a function of the at least one further parameter set during the pause in the first method recording.

5. The system according to claim 2, wherein the data processing device is programmed to, after pausing the first method recording prior to starting recording of the second recording portion of the first method recording, perform a second method recording associated with second method identification data different from the first method identification data,
in particular by programming the data processing device to,
receive, by means of the first communication device, after the execution of at least one pipetting operation associated with a second method, from the at least one second communication device recording data containing parameter values on the basis of which the at least one pipetting operation of the second method was configured and executed,
perform the second method recording by storing this recording data as a second method data set in the data storage device,
pause the execution of the second method recording in response to a user input occurring at the user interface device, and
continue the paused second method recording in response to user input at the user interface device, or terminate the paused second method recording in response to user input at the user interface device.

6. The system according to claim 5, which includes a first sample to be pipetted in the first method and includes a second sample to be pipetted in the second method that is not used in the first method.

7. The system according to claim 1, wherein the data processing device is programmed to, on the display of the user interface device,
display a screen page with a button, the pressing or touching of which starts a method recording.

8. The system according to claim 1, wherein the data processing device is programmed to, on the display of the user interface device,
display a screen page in which the title of at least one running, i.e. started and not terminated, method recording is displayed, in particular in which the titles of several running method recordings are listed,
display at least one information field indicating the status of the corresponding method recording, which may include: Recording is paused or Recording is running or Recording is terminated.

9. The system according to claim 1, wherein the data processing device is programmed to, on the display of the user interface device,
display a screen page in which the title of at least one running method recording is displayed, in particular in which the titles of several running method recordings are listed,
display at least one button, the pressing or touching of which selects the at least one previously paused method recording associated with the button;
and display a button, the pressing or touching of which resumes the selected paused method recording, in particular while at least one other method recording is paused.

10. The system according to claim 1, wherein the data processing device is programmed to, on the display of the user interface device,
display a screen page in which the title of at least one running method recording is displayed, in particular in which the titles of several running method recordings are listed,
display at least one button, the pressing or touching of which selects the at least one previously paused method recording associated with the button and causes the display of information about this method recording, which in particular contains information about, in each case preferably: the events of a method, in particular its time sequence, time data of the method, in particular time stamps of one or more events, in particular of the start, pause, and/or end of the method, the user of a method, the pipetting devices used and/or not used in the method.

11. The system according to claim 1, wherein the data processing device is programmed to provide at least one method data set of at least one terminated method recording as a file transferable to an external data processing device, or the contents of which are outputtable by printing, displaying on the screen, or transferring to an external data processing device of the system or to a server which is not part of the system.

12. The system according to claim 1, wherein the data processing device is programmed to,
obtain, by means of the user interface device, parameter values of user-definable pipetting parameters of at least one parameter set, which define the at least one pipetting operation of a method, in particular first, second, and still further methods, to be performed by means of the at least one pipetting device,
transmit, by means of the first communication device, these parameter values of the at least one parameter set to the at least one hand-held pipetting device in order to enable its configuration by means of these parameter values for carrying out at least one pipetting operation of the method.

13. The system according to claim 3, wherein the at least one pipetting device comprises a second data processing device programmed to,
obtain, by means of the second communication device, the parameter values of the at least one parameter set in order to configure the pipetting device by means of these parameter values for carrying out at least one pipetting operation of the method,
start, by means of the parameter values, the at least one pipetting operation configured, in particular by determining a start signal generated manually by the actuation of an actuating element, and
transmit, after completion or termination of the at least one pipetting operation configured by means of the parameter values, the parameter values used to configure this pipetting operation as recording data to the first communication device.

14. The system according to claim 1, wherein the data processing device is programmed to not store data, in particular recording data, transmitted from the second communication device to the first communication device as part of the first method recording in the first method data set during a recording pause of the first method recording,
and/or in particular to store the data, in particular recording data, transmitted from the second communication device to the first communication device during the recording pause of the first method recording, as a component of another, in particular a second, method recording in a further, in particular second, method data set assigned to the second method recording.

15. A method for using the system according to claim 1, comprising the steps of:
a) starting, in response to a user input, recording of a first recording portion of the first method recording, wherein a first subset of the first sequence of parameter sets of the first experiment portion is recorded and stored as a first method data set in the data storage device,
b) pausing, in response to a user input, the first method recording; and
c) selecting, in response to a user input and the first method identification data, the stored first method data set and continuing the first method recording the first experiment by starting the recording of a second recording portion of the first method recording in which the second subset of the first sequence of parameter sets of the second experiment portion is recorded.
